(19) United States
(12) Patent Application Publication
(10) Pub. No.: US 2015/0368357 A1
(43) Pub. Date: Dec. 24, 2015

(54) HIGHLY GALACTOSYLATED ANTI-HER2 ANTIBODIES AND USES THEREOF

(71) Applicant: LABORATOIRE FRANCAIS DU FRACTIONNEMENT ET DES BIOTECHNOLOGIES, Les Ulis (FR)

(72) Inventors: Harry M. Meade, Newton, MA (US); Li-How Chen, Acton, MA (US)

(21) Appl. No.: 14/767,120

(22) PCT Filed: Feb. 13, 2014
(86) PCT No.: PCT/IB2014/000711

§ 371 Date: Aug. 11, 2015

Related U.S. Application Data
(60) Provisional application No. 61/764,488, filed on Feb. 13, 2013.

(51) Int. Cl. C07K 16/32

(52) U.S. Cl. CPC C07K 16/32; C07K 2317/41; C07K 2317/14; C07K 2317/734; C07K 2317/732; C07K 2317/92

(57) ABSTRACT

In one aspect, the disclosure relates to highly galactosylated anti-HER2 antibodies and compositions thereof. In one aspect, the disclosure relates to populations of anti-HER2 antibodies with a high level of galactosylation, and compositions thereof. In one aspect, the disclosure relates to methods of production and use of highly galactosylated anti-HER2 antibodies and populations of anti-HER2 antibodies with a high level of galactosylation. In some embodiments the anti-HER-2 antibody is trastuzumab.

### RELATED APPLICATIONS

This application claims the benefit under 35 U.S.C. §119(e) of U.S. Provisional Application Ser. No. 61/764,488, entitled “Highly Galactosylated Anti-HER2 Antibodies and Uses Thereof,” filed on Feb. 13, 2013, the entire disclosure of which is incorporated by reference herein in its entirety.

### FIELD OF THE INVENTION

The present invention relates in part to field of anti-HER2 antibodies.

### BACKGROUND OF THE INVENTION

HER2 (Human Epidermal Growth Factor Receptor 2), also known as HER2/neu or ErbB2, is a member of the epidermal growth factor receptor family. HER2 is plasma-membrane bound receptor tyrosine kinase that can dimerize with itself and other members of the family of epidermal growth factor receptors (HER1, HER2, HER3 and HER4). Dimerization, in turn, results in the activation of a variety of intracellular pathways. HER2 is an oncogene that is overexpressed in a variety of cancers including breast, ovarian, stomach and uterine cancer. HER2 overexpression in cancer (“HER2+” cancer) is associated with poor prognosis.

HER2 is the target of the monoclonal antibody trastuzumab (Herceptin) which binds domain IV of the extracellular segment of the HER2/neu receptor. Trastuzumab was approved by the FDA in 1998 and has been used for the treatment of HER2+ breast cancer and HER2+ gastric cancer. However, trastuzumab is not therapeutically effective in a large number of patients with HER2+ cancers. In addition, treatment with trastuzumab has been associated with cardiac dysfunction and additional undesired side effects. Anti-HER2 antibodies with improved therapeutic properties are desired therefore.

### SUMMARY OF INVENTION

In one aspect, the disclosure relates to highly galactosylated anti-HER2 antibodies and compositions thereof. In one aspect, the disclosure relates to populations of anti-HER2 antibodies with a high level of galactosylation, and compositions thereof. In one aspect, the disclosure relates to methods of production and use of highly galactosylated anti-HER2 antibodies and populations of anti-HER2 antibodies with a high level of galactosylation. In some embodiments, the anti-HER2 antibody is trastuzumab.

In one aspect the disclosure provides an anti-HER2 antibody, wherein the antibody is highly galactosylated. In some embodiments, the antibody is highly fucosylated. In some embodiments, the antibody comprises mono-galactosylated N-glycans. In some embodiments, the antibody comprises bi-galactosylated N-glycans. In some embodiments, the heavy chain of the antibody comprises SEQ ID NO:1, and the light chain of the antibody comprises SEQ ID NO:2. In some embodiments, the antibody is trastuzumab. In some embodiments, the antibody is produced in mammary epithelial cells of a non-human mammal. In some embodiments, the antibody is produced in a transgenic non-human mammal. In some embodiments, the non-human mammal is a goat, sheep, bison, camel, cow, pig, rabbit, buffalo, horse, rat, mouse or llama. In some embodiments, the non-human mammal is a goat.

In one aspect the disclosure provides compositions of any of the antibodies disclosed herein, wherein the composition further comprises milk. In some embodiments, the composition further comprises a pharmaceutically-acceptable carrier.

In one aspect the disclosure provides a composition, comprising a population of antibodies, wherein the antibody is an anti-HER2 antibody, and wherein the level of galactosylation of the antibodies in the population is at least 50%. In some embodiments, the level of galactosylation of the antibodies in the population is at least 60%. In some embodiments, the level of galactosylation of the antibodies in the population is at least 70%. In some embodiments, the level of fucosylation of the antibodies in the population is at least 50%. In some embodiments, the level of fucosylation of the antibodies in the population is at least 60%. In some embodiments of any of the compositions provided herein, the population comprises antibodies that comprise mono-galactosylated N-glycans. In some embodiments of any of the compositions provided herein, the population comprises antibodies that comprise bi-galactosylated N-glycans. In some embodiments of any of the compositions provided herein, the ratio of the level of galactosylation of the antibodies in the population to the level of fucosylation of the antibodies in the population is between 1.0 and 1.4. In some embodiments of any of the compositions provided herein, at least 25% of the antibodies in the population comprise bi-galactosylated N-glycans and at least 25% of the antibodies in the population comprise mono-galactosylated N-glycans. In some embodiments of any of the compositions provided herein, the heavy chain of the antibody comprises SEQ ID NO:1, and the light chain of the antibody comprises SEQ ID NO:2. In some embodiments of any of the compositions provided herein, the antibody is trastuzumab. In some embodiments of any of the compositions provided herein, the antibody is produced in mammary epithelial cells of a non-human mammal. In some embodiments of any of the compositions provided herein, the antibody is produced in a transgenic non-human mammal. In some embodiments, the non-human mammal is a goat, sheep, bison, camel, cow, pig, rabbit, buffalo, horse, rat, mouse or llama. In some embodiments, the non-human mammal is a goat. In some embodiments, the composition further comprises milk. In some embodiments, the composition further comprises a pharmaceutically-acceptable carrier.

In some embodiments of any of the compositions provided herein, the population of antibodies has an increased level of complement dependent cytotoxicity (CDC) activity when compared to a population of antibodies not produced in mammary gland epithelial cells.

In some embodiments of any of the compositions provided herein, the population of antibodies has an increased level of antibody-dependent cellular cytotoxicity (ADCC) activity when compared to a population of antibodies not produced in mammary gland epithelial cells.

In some embodiments of any of the compositions provided herein, the population of antibodies has an increased ability to suppress HER2 activity in a subject when compared to a population of antibodies not produced in mammary gland epithelial cells.

In some embodiments of any of the compositions provided herein, the population of antibodies has an increased ability to bind HER2 when compared to a population of antibodies not produced in mammary gland epithelial cells.

In some embodiments of any of the compositions provided herein, the population of antibodies has an increased ability to suppress HER2 dimerization when compared to a population of antibodies not produced in mammary gland epithelial cells.

In some embodiments of any of the compositions provided herein, the population of antibodies not produced in mammary gland epithelial cells is produced in cell culture.

In some embodiments of any of the compositions provided herein, the level of galactosylation of the antibodies not produced in mammary gland epithelial cells is 50% or lower when compared to the level of galactosylation of the antibodies produced in mammary gland epithelial cells. In some embodiments of any of the compositions provided herein, the level of galactosylation of the antibodies not produced in mammary gland epithelial cells is 30% or lower when compared to the level of galactosylation of the antibodies produced in mammary gland epithelial cells. In some embodiments of any of the compositions provided herein, the level of galactosylation of the antibodies not produced in mammary gland epithelial cells is 10% or lower when compared to the level of galactosylation of the antibodies produced in mammary gland epithelial cells.

In one aspect, the disclosure provides a method for producing a population of antibodies, comprising: expressing the population of antibodies in mammary gland epithelial cells of a non-human mammal such that a population of antibodies is produced, wherein the antibody is an anti-HER2 antibody, wherein the level of galactosylation of the antibodies in the population is at least 50%. In some embodiments, the mammary gland epithelial cells are in culture and are transfected with a nucleic acid that comprises a sequence that encodes the antibody. In some embodiments, the mammary gland epithelial cells are in a non-human mammal engineered to express a nucleic acid that comprises a sequence that encodes the antibody in its mammary gland. In some embodiments, the nucleic acid comprises SEQ ID NO:3 and SEQ ID NO:4. In some embodiments, the mammary gland epithelial cells are goat, sheep, bison, camel, cow, pig, rabbit, buffalo, horse, rat, mouse or llama mammary gland epithelial cells. In some embodiments, the mammary gland epithelial cells are goat mammary gland epithelial cells.

In one aspect, the disclosure provides mammary gland epithelial cells that produce any of the antibodies, population of antibodies, or compositions disclosed herein.

In one aspect, the disclosure provides a transgenic non-human mammal comprising any of the mammary gland epithelial cells disclosed herein.

In one aspect, the disclosure provides a method comprising administering any of the antibodies, population of antibodies, or compositions disclosed herein to a subject in need thereof. In some embodiments, the subject has cancer. In some embodiments, the cancer is a HER2+ cancer. In some embodiments, the HER2+ cancer is breast, ovarian, stomach or uterine cancer.

In one aspect, the disclosure provides a monoclonal anti-HER2 antibody composition comprising monoclonal anti-HER2 antibodies having glycan structures on the Fc glycosylation sites (Asn297, EU numbering), wherein said glycan structures have a galactose content of more than 60%.

### BRIEF DESCRIPTION OF THE DRAWINGS

The drawings are first described. It is to be understood that the drawings are exemplary and not required for enablement of the invention.

FIGS. 1A and 1B show representative oligosaccharide signatures of N-glycans of populations of highly galactosylated trastuzumab antibodies from goat #2.

FIG. 2 shows an oligosaccharide signature of N-glycans of a population of highly galactosylated trastuzumab antibodies from goat #1 at day 7 of lactation

FIG. 3 shows an oligosaccharide signature of N-glycans of a population of highly galactosylated trastuzumab antibodies from goat #1 at day 15 of lactation.

FIG. 4 shows an oligosaccharide signature of N-glycans of a population of highly galactosylated trastuzumab antibodies from goat #1 at day 30 of lactation.

FIG. 5 shows a summary of the percentages of N-glycan oligosaccharides of populations of highly galactosylated trastuzumab antibodies from goat #1 at various days of lactation.

FIG. 6 shows an oligosaccharide signature of N-glycans of a population of highly galactosylated trastuzumab antibodies from goat #2 at day 7 of the first lactation.

FIG. 7 shows a summary of the percentages of N-glycan oligosaccharides of a population of highly galactosylated trastuzumab antibodies from goat #2 at day 7 of the first lactation.

FIG. 8 shows a summary of the percentages of N-glycan oligosaccharides of a population of highly galactosylated trastuzumab antibodies from goat #2 at days 15, 49, 84, 112 of the first lactation.

FIG. 9 shows a summary of the percentages of N-glycan oligosaccharides of populations of highly galactosylated trastuzumab from goat #3 at day 7 of lactation and goat #4 at day 3/4 of lactation.

FIG. 10 shows a summary of the percentages of N-glycan oligosaccharides of populations of highly galactosylated trastuzumab from goat #5 at day 3 of lactation and goat 6 at days 5, 6, and 7 of lactation.

FIG. 11 shows a summary of the percentages of N-glycan oligosaccharides of populations of highly galactosylated trastuzumab from goat #2 at days 8, 15, and 29 of the second lactation.

FIG. 12 shows a summary of the percentages of N-glycan oligosaccharides of commercial Herceptin®/trastuzumab.

FIG. 13 shows a summary comparing the sialic acid and mannose modifications and predominant forms of trastuzumab produced by goat #2 at various days of first lactation (NL1) or second lactation (NL2).

FIG. 14 shows a summary of the sialic acid and mannose modifications and predominant forms of trastuzumab produced in goats #1-6.

FIG. 15 shows that transgenically produced trastuzumab antibodies bind to SK-BR-3 cells known to express HER2.

FIG. 16 shows that transgenically produced trastuzumab antibodies have similar binding affinities to SK-BR-3 cells as compared to commercial Herceptin®/trastuzumab.

FIG. 17 shows that transgenically produced trastuzumab antibodies interact with CD16 expressed on NK cells.

FIG. 18 shows that transgenically produced trastuzumab antibodies have enhanced antibody-dependent cellular cytotoxicity (ADCC) compared to commercial Herceptin®/trastuzumab.

FIG. 19 shows that transgenically produced trastuzumab antibodies reduce proliferation of BT-474 cells.

### DETAILED DESCRIPTION OF INVENTION

In one aspect, the disclosure provides anti-HER2 antibodies wherein the antibody is highly galactosylated. Anti-HER2 antibodies bind HER2 and anti-HER2 antibodies have been used as a therapeutic in a variety of cancers characterized by the overexpression of HER2 (HER2+ cancers). In some embodiments, the anti-HER2 antibody that is highly galactosylated is trastuzumab.

In some embodiments, the anti-HER2 antibody that is highly galactosylated includes a heavy chain which comprises SEQ ID NO:1. In some embodiments, the anti-HER2 antibody that is highly galactosylated includes a light chain which comprises SEQ ID NO:2. In some embodiments, the anti-HER2 antibody that is highly galactosylated includes a heavy chain which comprises SEQ ID NO:1 and a light chain which comprises SEQ ID NO:2. In some embodiments, the anti-HER2 antibody that is highly galactosylated includes a heavy chain which consists of SEQ ID NO:1. In some embodiments, the anti-HER2 antibody that is highly galactosylated includes a light chain that consists of SEQ ID NO:2. In some embodiments, the anti-HER2 antibody that is highly galactosylated includes a heavy chain which consists of SEQ ID NO:1 and a light chain that consists of SEQ ID NO:2. In some embodiments, the anti-HER2 antibody that is highly galactosylated is trastuzumab.

The heavy chain of trastuzumab is provided in SEQ ID NO:1:

MEFGLSWLFLVAILKGVQCEVQLVESGGGLVQPGGSLRLSCAASGFNIKDT
YIHWVRQAPGKGLEWVARIYPTNGYTRYADSVKGRFTISADTSKNTAYLQM
NSLRAEDTAVYYCSRWGGDGFYAMDYWGQGTLVTVSSASTKGPSVFPLAPS
SKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPE
LLGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYT
QKSLSLSPGK

The light chain of trastuzumab is provided in SEQ ID NO:2:

MDMRVPAQLLGLLLLWLRGARCDIQMTQSPSSLSASVGDRVTITCRASQDV
NTAVAWYQQKPGKAPKLLIYSASFLYSGVPSRFSGSRSGTDFTLTISSLQP
EDFATYYCQQHYTTPPTFGQGTKVEIKRTVAAPSVFIFPPSDEQLKSGTAS
VVCLLNNFYPREAKVQWKVDNALQSGNSQESVTEQDSKDSTYSLSSTLTLS
KADYEKHKVYACEVTHQGLSSPVTKSFNRGEC

It should further be appreciated that in some embodiments, the disclosure also includes antibodies that are based on the sequence of trastuzumab but that include mutations that provide the antibodies with additional beneficial desired properties related to bioavailability, stability etc.

In one aspect, the disclosure provides anti-HER2 antibodies wherein the antibody is highly galactosylated. In some embodiments, the disclosure provides anti-HER2 antibodies, wherein the antibody is highly fucosylated. In some embodiments, the disclosure provides anti-HER2 antibodies, wherein the antibody is highly galactosylated and highly fucosylated. In some embodiments, the highly galactosylated antibody comprises one or more mono-galactosylated N-glycans. In some embodiments, the highly galactosylated antibody comprises bi-galactosylated N-glycans.

In one aspect, the disclosure provides a monoclonal anti-HER2 antibody composition comprising monoclonal antibodies having on the Fc glycosylation sites (Asn 297, EU numbering) glycan structures, wherein said glycan structures have a galactose content more than 60%. In one embodiment the anti-HER2 monoclonal antibodies are purified. The “EU numbering system” or “EU index” is generally used when referring to a residue in an immunoglobulin heavy chain constant region (e.g., the EU index reported in Kabat et al., Sequences of Proteins of Immunological Interest, 5th ed., Public Health Service, National Institutes of Health, Bethesda, Md. (1991) expressly incorporated herein by reference). The typical glycosylated residue position in an antibody is the asparagine at position 297 according to the EU numbering system (“Asn297”).

It should be appreciated that any of the anti-HER2 monoclonal antibodies disclosed herein may be partially or completely purified.

Antibodies can be glycosylated with an N-glycan at the Fc-gamma glycosylation site in the heavy chain (Asn297) of the Fc region. Generally, antibodies include two heavy chains and each antibody therefore can have two Fc-gamma N-glycans. A variety of glycosylation patterns have been observed at the Fc gamma glycosylation site and the oligosaccharides found at this site include galactose, N-acetylglucosamine (GlcNac), mannose, sialic acid, N-acetylneuraminic acid (NeuAc or NANA), N-glycolylneuraminic (NGNA) and fucose. N-glycans found at the Fc gamma glycosylation site generally have a common core structure consisting of an unbranched chain of a first N-acetylglucosamine (GlcNAc), which is attached to the asparagine of the antibody, a second GlcNAc that is attached to the first GlcNac and a first mannose that is attached to the second GlcNac. Two additional mannoses are attached to the first mannose of the GlcNAc-GlcNAc-mannose chain to complete the core structure and providing two “arms” for additional glycosylation. In addition, fucose residues may be attached to the N-linked first GlcNAc.

The two arm core structure is also referred to as the “antenna”. The most common type of glycosylation of the “arms” of the N-glycan motifs found in plasma antibodies is of the complex type, i.e., consisting of more than one type of monosaccharide. In the biosynthetic route to this N-glycan motif, several GlcNAc transferases attach GlcNAc residues to the mannoses of the glycan core, which can be further extended by galactose, sialic acid and fucose residues. This glycosylation motif is called “complex” structure.

A second glycosylation motif found on the “arms” of the N-glycan core structure is a “high-mannose” motif, which is characterized by additional mannoses (attached either as branched or unbranded chains).

A third glycosylation motif is a hybrid structure in which one of the arms is mannose substituted while the other arm is complex.

A “galactosylated” antibody, as used herein, refers to any antibody that has at least one galactose monosaccharide in one of its N-glycans. Galactosylated antibodies include antibodies where the two N-glycans each have complex type motifs on each of the arms of the N-glycan motifs, antibodies where the two N-glycans have a complex type motif on only one of the arms of the N-glycan motifs, antibodies that have one N-glycan with complex type motifs on each of the arms of the N-glycan, and antibodies that have one N-glycan with a complex type motif on only one of the arms of the N-glycan motifs. Antibodies that include at least one galactose monosaccharide include antibodies with N-glycans such as G1 (one galactose), G1F (one galactose, one fucose), G2 (two galactoses) and G2F (two galactoses, one fucose). In addition, the N-glycan that includes at least one galactose monosaccharide can be sialylated or not sialylated. It should further be appreciated that the N-glycans may also contain additional galactose residues, such as alpha-Gal, in one or more arms of the complex glycan motif, potentially resulting in an N-glycan with four galactose moieties.

A “highly galactosylated” antibody, as used herein, refers to an antibody that includes at least two galactose monosaccharides in the N-glycan motifs. Highly galactosylated antibodies include antibodies where the two N-glycans each have complex type motifs on each of the arms of the N-glycan motifs, antibodies where the two N-glycans have a complex type motif on only one of the arms of the N-glycan motifs, and antibodies that have one N-glycan with a complex type motif on each of the arms of the N-glycan. Thus, highly galactosylated antibodies include antibodies in which both N-glycans each include one galactose in the glycan motif (e.g., G1 or G1F), antibodies that include at least one N-glycan with two galactoses in the glycan motif (e.g., G2 or G2F), and antibodies with 3 or 4 galactoses in the glycan motif (e.g., (i) one N-glycan with a G1 glycan motif and one N-glycan with a G2 or G2F glycan motif or (ii) two N-glycan with G2 or G2F). In some embodiments, the highly galactosylated antibody includes at least three galactose monosaccharides in the glycan motifs. In some embodiments, the highly galactosylated antibody includes at least four galactose monosaccharides in the glycan motifs.

The glycosylation pattern of the N-glycans can be determined by a variety of methods known in the art. For example, methods of analyzing carbohydrates on proteins have been described in U.S. Patent Applications US 2006/0057638 and US 2006/0127950. The methods of analyzing carbohydrates on proteins are incorporated herein by reference.

In some embodiments, the highly galactosylated antibody is produced in mammary epithelial cells of a non-human mammal. In some embodiments, the antibody is produced in a transgenic non-human mammal. In some embodiments, the non-human mammal is a goat, sheep, bison, camel, cow, pig, rabbit, buffalo, horse, rat, mouse or llama. In some embodiments, the non-human mammal is a goat.

In some embodiments, the highly glycosylated antibody is produced in cells other than in mammary epithelial cells of a non-human mammal. In some embodiments, the antibody is produced in cells other than in mammary epithelial cells of a non-human mammal and modified after production to increase the number of galactose groups on the N-glycan (e.g., through the action of enzymes such as transferases).

In one aspect, the disclosure provides compositions comprising highly galactosylated antibodies. In some embodiments, the composition comprising highly galactosylated antibodies further comprises milk. In some embodiments, the composition comprising highly galactosylated antibodies further comprises a pharmaceutically-acceptable carrier.

In one aspect, the disclosure provides compositions comprising monoclonal anti-HER2 antibody compositions having on the Fc glycosylation sites (Asn 297, EU numbering) glycan structures, wherein said glycan structures of the monoclonal antibodies have a galactose content more than 60%. In some embodiments, the composition comprising monoclonal anti-HER2 antibody compositions further comprises milk. In some embodiments, the composition comprising monoclonal anti-HER2 antibody compositions further comprises a pharmaceutically-acceptable carrier.

### Populations of Antibodies

In one aspect, the disclosure provides a composition comprising a population of antibodies, wherein the antibody is an anti-HER2 antibody, and wherein the level of galactosylation of the antibodies in the population is at least 50%. In some embodiments, the level of galactosylation of the antibodies in the population is at least 60%. In some embodiments, the level of galactosylation of the antibodies in the population is at least 70%. In some embodiments, the level of fucosylation of the antibodies in the population is at least 50%. In some embodiments, the level of fucosylation of the antibodies in the population is at least 60%. In some embodiments, the population comprises antibodies that comprise mono-galactosylated N-glycans. In some embodiments, the population comprises antibodies that comprise bi-galactosylated N-glycans. In some embodiments, the ratio of the level of galactosylation of the antibodies in the population to the level of fucosylation of the antibodies in the population is between 1.0 and 1.4. In some embodiments, at least 25% of the antibodies in the population comprise bi-galactosylated N-glycans and at least 25% of the antibodies in the population comprise mono-galactosylated N-glycans.

In some embodiments, the anti-HER2 antibody of the populations of antibodies with a high level of galactosylation is trastuzumab. In some embodiments, the anti-HER2 antibody of the populations of antibodies with a high level of galactosylation comprises a heavy chain which comprises SEQ ID NO:1. In some embodiments, the anti-HER2 antibody of the populations of antibodies with a high level of galactosylation comprises a light chain which comprises SEQ ID NO:2. In some embodiments, the anti-HER2 antibody of the populations of antibodies with a high level of galactosylation comprises a heavy chain which comprises SEQ ID NO:1 and a light chain which comprises SEQ ID NO:2. In some embodiments, the anti-HER2 antibody of the populations of antibodies with a high level of galactosylation comprises a heavy chain which consists of SEQ ID NO:1. In some embodiments, the anti-HER2 antibody of the populations of antibodies with a high level of galactosylation comprises a light chain that consists of SEQ ID NO:2. In some embodiments, the anti-HER2 antibody of the populations of antibodies with a high level of galactosylation comprises a heavy chain which consists of SEQ ID NO:1 and a light chain that consists of SEQ ID NO:2. In some embodiments, the anti-HER2 antibody of the populations of antibodies with a high level of galactosylation is trastuzumab.

The biosynthesis of N-glycans is not regulated by a template, as is the case with proteins, but is mainly dependent on the expression and activity of specific glycosyltransferases in a cell. Therefore, a glycoprotein, such as an antibody Fc domain, normally exists as a heterogeneous population of glycoforms which carry different glycans on the same protein backbone.

A population of anti-HER2 antibodies that is highly galactosylated is a population of antibodies wherein the level of galactosylation of the antibodies in the population is at least 50%, at least 60%, at least 70%, at least 80%, at least 90%, up to 100% of galactosylation. In some embodiments of the population of antibodies that is highly galactosylated, the level of galactosylation of the antibodies in the population is at least 60%.

The level of galactosylation as used herein is determined by the following formula:

$${\sum }_{l = 1}^{n} ( \frac{( number of Gal )}{( number of A )} * ( \% relative Area ) )$$

wherein:
n represents the number of analyzed N-glycan peaks of a chromatogram, such as a Normal-Phase High Performance Liquid Chromatography (NP HPLC) spectrum
“number of Gal” represents the number of Galactose motifs on the antennae of the glycan corresponding to the peak, and
“number of A” corresponds to the number of N-acetylglucosamine motifs on the antennae of the glycan form corresponding to the peak (excluding the two N-acetylglucosamine motifs of the core structure), and
“% relative Area” corresponds to % of the Area under the corresponding peak

The level of galactosylation of antibodies in a population of antibodies can be determined, for instance, by releasing the N-glycans from the antibodies, resolving the N-glycans on a chromatogram, identifying the oligosaccharide motif of the N-glycan that corresponds to a specific peak, determining the peak intensity and applying the data to the formula provided above (See also the experimental section provided herein).

Anti-HER2 antibodies that are galactosylated include antibodies that are mono-galactosylated N-glycans and bi-galactosylated N-glycans.

In some embodiments of the population of antibodies that are highly galactosylated, the population comprises antibodies that comprise mono-galactosylated N-glycans, which may or may not be sialylated. In some embodiments of the population of antibodies that is highly galactosylated, at least 1%, at least 5%, at least 10%, at least 15%, at least 20%, at least 25%, at least 30%, at least 40%, at least 50%, at least 60%, at least 70%, at least 80%, at least 90%, up to 100% of the antibody N-glycans comprise mono-galactosylated N-glycans. In some embodiments of the population of antibodies that is highly galactosylated, at least 25% of the antibodies comprise mono-galactosylated N-glycans.

In some embodiments of the population of antibodies that are highly galactosylated, the population comprises antibodies that comprise bi-galactosylated N-glycans, which may or may not be sialylated. In some embodiments of the population of antibodies that is highly galactosylated, at least 1%, at least 5%, at least 10%, at least 15%, at least 20%, at least 25%, at least 30%, at least 40%, at least 50%, at least 60%, at least 70%, at least 80%, at least 90%, up to 100% of the antibody N-glycans comprise bi-galactosylated N-glycans. In some embodiments of the population of antibodies that is highly galactosylated, at least 25% of the antibodies comprise bi-galactosylated N-glycans.

In some embodiments of the population of antibodies that is highly galactosylated, the population comprises antibodies that comprise mono-galactosylated N-glycans, which may or may not be sialylated, and antibodies that comprise bi-galactosylated N-glycans, which may or may not be sialylated. In some embodiments of the population of antibodies that is highly galactosylated, at least 1%, at least 5%, at least 10%, at least 15%, at least 20%, at least 25%, at least 30%, at least 40%, at least 50%, at least 60%, at least 70%, at least 80%, at least 90%, up to 99% of the antibody N-glycans comprise mono-galactosylated N-glycans, and at least 1%, at least 5%, at least 10%, at least 15%, at least 20%, at least 25%, at least 30%, at least 40%, at least 50%, at least 60%, at least 70%, at least 80%, at least 90%, up to 99% of the antibody N-glycans comprise bi-galactosylated N-glycans. In some embodiments of the population of antibody N-glycans that is highly galactosylated, at least 25% of the antibody N-glycans comprise mono-galactosylated N-glycans and at least 25% of the antibodies comprise bi-galactosylated N-glycans.

In some embodiments of the population of antibodies that is highly galactosylated, the population comprises antibodies that are highly fucosylated. A population of antibodies that is highly fucosylated is a population of antibodies wherein the level of fucosylation of the antibody N-glycans in the population is at least 50%, at least 60%, at least 70%, at least 80%, at least 90%, up to 100% of fucosylation. In some embodiments in the population of antibodies that is highly galactosylated, the level of fucosylation of the antibody N-glycans is at least 50%.

The level of fucosylation as used herein is determined by the following formula:

$${\sum }_{i = 1}^{n} ( number of Fucose ) * ( \% relative Area )$$

wherein:
n represents the number of analyzed N-glycan peaks of a chromatogram, such as a Normal-Phase High Performance Liquid Chromatography (NP HPLC) spectrum, and
“number of Fucose” represents the number of Fucose motifs on the glycan corresponding to the peak, and
“% relative Area” corresponds to % of the Area under the corresponding peak containing the Fucose motif.

Antibodies that are fucosylated include antibodies that have at least one fucose monosaccharide in one of its N-glycans. Antibodies that are fucosylated include antibodies that have a fucose monosaccharide in each of its N-glycans.

In some embodiments, the population of anti-HER2 antibodies disclosed herein relates to a population wherein the level of galactosylation of the antibody N-glycans in the population is at least 50% and the level of fucosylation of the antibodies in the population is at least 50%. In some embodiments, the population of antibodies disclosed herein relates to a population wherein the level of galactosylation of the antibody N-glycans in the population is at least 50%, and the level of fucosylation of the antibody N-glycans in the population is at least 50%, at least 60%, at least 70%, at least 80%, at least 90%, up to 100%. In some embodiments, the population of antibodies disclosed herein relates to a population wherein the level of galactosylation of the antibody N-glycans in the population is at least 60%, and the level of fucosylation of the antibody N-glycans in the population is at least 50%, at least 60%, at least 70%, at least 80%, at least 90%, up to 100%. In some embodiments, the population of antibodies disclosed herein relates to a population wherein the level of galactosylation of the antibody N-glycans in the population is at least 70%, and the level of fucosylation of the antibody N-glycans in the population is at least 50%, at least 60%, at least 70%, at least 80%, at least 90%, up to 100%. In some embodiments, the population of antibodies disclosed herein relates to a population wherein the level of galactosylation of the antibody N-glycans in the population is at least 80%, and the level of fucosylation of the antibody N-glycans in the population is at least 50%, at least 60%, at least 70%, at least 80%, at least 90%, up to 100%. In some embodiments, the population of antibodies disclosed herein relates to a population wherein the level of galactosylation of the antibody N-glycans in the population is at least 90%, and the level of fucosylation of the antibody N-glycans in the population is at least 50%, at least 60%, at least 70%, at least 80%, at least 90%, up to 100%. In some embodiments, the population of antibodies disclosed herein relates to a population wherein the level of galactosylation of the antibody N-glycans in the population is up to 100% and the level of fucosylation of the antibody N-glycans in the population is at least 50%, at least 60%, at least 70%, at least 80%, at least 90%, up to 100%.

In one aspect, the disclosure relates to a composition comprising a population of anti-HER2 antibodies with a specific ratio of the percentage of antibody N-glycans in the population that are galactosylated at the Fc-gamma-glycosylation site to the percentage of antibody N-glycans in the population that are fucosylated at the Fc-gamma-glycosylation site. In some embodiments, the disclosure relates to a composition comprising a population of antibodies wherein the ratio of the level of galactosylation of the antibody N-glycans in the population to the level of fucosylation of the antibody N-glycans in the population is between 0.5 and 2.5, between 0.6 and 2.0, between 0.7 and 1.8, between 0.8 and 1.6, or between 1.0 and 1.4. In some embodiments, the disclosure relates to a composition comprising a population of antibodies wherein the ratio of the level of galactosylation of the antibody N-glycans in the population to the level of fucosylation of the antibody N-glycans in the population is between 1.0 and 1.4, for example 1.2.

In some embodiments, the population of anti-HER2 antibodies with a high level of galactosylation is produced in mammary epithelial cells of a non-human mammal. In some embodiments, the population of anti-HER2 antibodies is produced in a transgenic non-human mammal. In some embodiments, the non-human mammal is a goat, sheep, bison, camel, cow, pig, rabbit, buffalo, horse, rat, mouse or llama. In some embodiments, the non-human mammal is a goat.

In some embodiments, the population of anti-HER2 antibodies with a high level of galactosylation is produced in cells other than mammary epithelial cells of a non-human mammal. In some embodiments, the population of anti-HER2 antibodies is modified after production in cells other than mammary epithelial cells of a non-human mammal to increase the number of galactose groups in the population of antibodies (e.g., through the action of enzymes such as transferases).

In one aspect, the disclosure provides compositions comprising populations of anti-HER2 antibodies with a high level of galactosylation. In some embodiments, the composition comprising anti-HER2 antibodies with a high level of galactosylation further comprises milk. In some embodiments, the composition comprising anti-HER2 antibodies with a high level of galactosylation further comprises a pharmaceutically-acceptable carrier.

### Production of Populations of Antibodies

In one aspect, the disclosure provides compositions comprising populations of anti-HER2 antibodies with high levels of galactosylation (e.g., at least 60%), wherein the population of antibodies is produced in mammary epithelial cells of a non-human mammal, and wherein the population of antibodies has an increased level of galactosylation when compared to the population of antibodies not produced in mammary gland epithelial cells. In some embodiments, the population of antibodies not produced in mammary gland epithelial cells is produced in cell culture. As used herein, antibodies “produced in cell culture” when compared to antibodies produced in mammary epithelial cells, refers to antibodies produced in standard production cell lines (e.g., CHO cells) but excluding mammary epithelial cells. In some embodiments, the level of galactosylation of the antibodies not produced in mammary gland epithelial cells is 90% or lower, 80% or lower, 70% or lower, 60% or lower, 50% or lower, 40% or lower, 30% or lower, 20% or lower, 10% or lower when compared to the level of galactosylation of the antibodies produced in mammary epithelial cells of a non-human mammal. In some embodiments, the level of galactosylation of the antibodies not produced in mammary gland epithelial cells is 50% or lower when compared to the level of galactosylation of the antibodies produced in mammary epithelial cells of a non-human mammal. In some embodiments, the level of galactosylation of the antibodies not produced in mammary gland epithelial cells is 30% or lower when compared to the level of galactosylation of the antibodies produced in mammary epithelial cells of a non-human mammal. In some embodiments, the level of galactosylation of the antibodies not produced in mammary gland epithelial cells is 10% or lower when compared to the level of galactosylation of the antibodies produced in mammary epithelial cells of a non-human mammal.

In one aspect, the disclosure provides compositions comprising populations of anti-HER2 antibodies with high levels of fucosylation (e.g., at least 60%), wherein the population of antibodies is produced in mammary epithelial cells of a non-human mammal, and wherein the population of antibodies has an increased level of fucosylation when compared to the population of antibodies not produced in mammary gland epithelial cells. In some embodiments, the population of antibodies not produced in mammary gland epithelial cells is produced in cell culture. As used herein, antibodies “produced in cell culture” when compared to antibodies produced in mammary epithelial cells, refers to antibodies produced in standard production cell lines (e.g., CHO cells) but excluding mammary epithelial cells. In some embodiments, the level of fucosylation of the antibodies not produced in mammary gland epithelial cells is 90% or lower, 80% or lower, 70% or lower, 60% or lower, 50% or lower, 40% or lower, 30% or lower, 20% or lower, 10% or lower when compared to the level of fucosylation of the antibodies produced in mammary epithelial cells of a non-human mammal. In some embodiments, the level of fucosylation of the antibodies not produced in mammary gland epithelial cells is 50% or lower when compared to the level of fucosylation of the antibodies produced in mammary epithelial cells of a non-human mammal. In some embodiments, the level of fucosylation of the antibodies not produced in mammary gland epithelial cells is 30% or lower when compared to the level of fucosylation of the antibodies produced in mammary epithelial cells of a non-human mammal. In some embodiments, the level of fucosylation of the antibodies not produced in mammary gland epithelial cells is 10% or lower when compared to the level of fucosylation of the antibodies produced in mammary epithelial cells of a non-human mammal.

In one aspect, the disclosure provides compositions comprising populations of anti-HER2 antibodies with high levels of galactosylation and fucosylation, wherein the population of antibodies is produced in mammary epithelial cells of a non-human mammal, and wherein the population of antibodies has an increased level of galactosylation and fucosylation when compared to the population of antibodies not produced in mammary gland epithelial cells.

### Antibodies

In some embodiments, the term “antibody” refers to a glycoprotein comprising at least two heavy (H) chains and two light (L) chains. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. In some embodiments, the antigen is HER2. The constant regions of the antibodies may mediate the binding of the immunoglobulin to host tissues or factors, including various cells of the immune system (e.g., effector cells) and the first component (C1q) of the classical complement system. Formation of a mature functional antibody molecule can be accomplished when two proteins are expressed in stoichiometric quantities and self-assemble with the proper configuration.

The term “antibodies” is also meant to encompass antigen-binding fragments thereof. Methods for making antibodies and antigen-binding fragments are well known in the art (see, e.g., Sambrook et al, “Molecular Cloning: A Laboratory Manual” (2nd Ed.), Cold Spring Harbor Laboratory Press (1989); Lewin, “Genes IV”, Oxford University Press, New York, (1990), and Roitt et al., “Immunology” (2nd Ed.), Gower Medical Publishing, London, New York (1989), WO2006/040153, WO2006/122786, and WO2003/002609). As used herein, an “antigen-binding fragment” of an antibody refers to one or more portions of an antibody that retain the ability to specifically bind to an antigen, e.g., HER2. It has been shown that the antigen-binding function of an antibody can be performed by fragments of a full-length antibody. Examples of binding fragments encompassed within the term “antigen-binding fragment” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546) which consists of a VH domain; and (vi) an isolated complementarity determining region (CDR). Furthermore, although the two domains of the Fv fragment, V and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional procedures, such as proteolytic fragmentation procedures, as described in J. Goding, Monoclonal Antibodies: Principles and Practice, pp 98-118 (N.Y. Academic Press 1983), which is hereby incorporated by reference as well as by other techniques known to those with skill in the art. The fragments are screened for utility in the same manner as are intact antibodies.

In some embodiments the antibodies are of the isotype IgG, IgA or IgD. In further embodiments, the antibodies are selected from the group consisting of IgG1, IgG2, IgG3, IgG4, IgM, IgA1, IgA2, IgAsec, IgD, IgE or has immunoglobulin constant and/or variable domain of IgG1, IgG2, IgG3, IgG4, IgM, IgA1, IgA2, IgAsec, IgD or IgE. In other embodiments, the antibodies are bispecific or multispecific antibodies. According to an alternative embodiment, the antibodies of the present disclosure can be modified to be in the form of a bispecific antibody, or a multispecific antibody. The term “bispecific antibody” is intended to include any agent, e.g., a protein, peptide, or protein or peptide complex, which has two different binding specificities which bind to, or interact with (a) a cell surface antigen and (b) an Fc receptor on the surface of an effector cell. The term “multispecific antibody” is intended to include any agent, e.g., a protein, peptide, or protein or peptide complex, which has more than two different binding specificities which bind to, or interact with (a) a cell surface antigen, (b) an Fc receptor on the surface of an effector cell, and (c) at least one other component. Accordingly, the disclosure includes, but is not limited to, bispecific, trispecific, tetraspecific, and other multispecific antibodies which are directed to cell surface antigens, and to Fc receptors on effector cells. The term “bispecific antibodies” further includes diabodies. Diabodies are bivalent, bispecific antibodies in which the VH and VL domains are expressed on a single polypeptide chain, but using a linker that is too short to allow for pairing between the two domains on the same chain, thereby forcing the domains to pair with complementary domains of another chain and creating two antigen-binding sites (see e.g., Holliger, P., et al. (1993) Proc. Natl. Acad. Sci. USA 90:6444-6448; Poijak, R. J., et al. (1994) Structure 2:1121-1123).

The term “antibodies” also encompasses different types of antibodies, e.g., recombinant antibodies, monoclonal antibodies, humanized antibodies or chimeric antibodies, or a mixture of these.

In some embodiments, the antibodies are recombinant antibodies. The term “recombinant antibody”, as used herein, is intended to include antibodies that are prepared, expressed, created or isolated by recombinant means, such as antibodies isolated from an animal that is transgenic for another species' immunoglobulin genes, antibodies expressed using a recombinant expression vector transfected into a host cell, antibodies isolated from a recombinant, combinatorial antibody library, or antibodies prepared, expressed, created or isolated by any other means that involves splicing of immunoglobulin gene sequences to other DNA sequences.

In yet other embodiments, the antibodies can be chimeric or humanized antibodies. As used herein, the term “chimeric antibody” refers to an antibody that combines parts of a non-human (e.g., mouse, rat, rabbit) antibody with parts of a human antibody. As used herein, the term “humanized antibody” refers to an antibody that retains only the antigen-binding CDRs from the parent antibody in association with human framework regions (see, Waldmann, 1991, Science 252:1657). Such chimeric or humanized antibodies retaining binding specificity of the murine antibody are expected to have reduced immunogenicity when administered in vivo for diagnostic, prophylactic or therapeutic applications according to the disclosure.

In certain embodiments, the antibodies are human antibodies. The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo). Human antibodies are generated using transgenic mice carrying parts of the human immune system rather than the mouse system. Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals results in the production of fully human antibodies to the antigen of interest. Following immunization of these mice (e.g., XenoMouse (Abgenix), HuMAb mice (Medarex/GenPharm)), monoclonal antibodies are prepared according to standard hybridoma technology. These monoclonal antibodies have human immunoglobulin amino acid sequences and therefore will not provoke human anti-mouse antibody (HAMA) responses when administered to humans. The human antibodies, like any of the antibodies provided herein can be monoclonal antibodies.

In some embodiments, the antibody is a full-length antibody. In some embodiments the full-length antibody comprises a heavy chain and a light chain. In some embodiments, the antibody is an anti-HER2 antibody. In some embodiments, the heavy chain comprises SEQ ID NO:1 and the light chain comprises SEQ ID NO:2. In some embodiments, the antibody is trastuzumab.

### CDC Activity

In one aspect, the compositions comprising populations of anti-HER2 antibodies with high levels of galactosylation (e.g., at least 60%) have high complement dependent cytotoxicity (CDC) activity. In one aspect, the compositions comprising populations of anti-HER2 antibodies with high levels of galactosylation have high antibody-dependent cellular cytotoxicity (ADCC) activity. In some embodiments, the compositions comprising populations of anti-HER2 antibodies with high levels of galactosylation have high complement dependent cytotoxicity (CDC) activity and have high antibody-dependent cellular cytotoxicity (ADCC) activity.

In some embodiments, the population of anti-HER2 antibodies with high levels of galactosylation has an increased level of complement dependent cytotoxicity (CDC) activity when compared to a population of antibodies that have low levels of galactosylation. In some embodiments, the population of antibodies with high levels of galactosylation and the population of antibodies that have low levels of galactosylation are directed to the same antigen epitope. In some embodiments, the population of antibodies that is highly galactosylated and the population of antibodies that have low levels of galactosylation are encoded by the same nucleic acid. In some embodiments, the nucleic acid encodes the antibody trastuzumab.

A population of antibodies that has low levels of galactosylation (is “low galactose”), as used herein, refers to a population of antibodies wherein the level of galactosylation of the antibodies in the population is less than 50%, less than 40%, less than 30%, less than 20%, less than 10%, down to 0%.

In some embodiments, the CDC activity of a population of antibodies with high levels of galactosylation is at least 1.1 times higher, at least 1.2 times higher, at least 1.3 times higher, at least 1.4 times higher, at least 1.5 times higher, at least 1.6 times higher, at least 1.7 times higher, at least 1.8 times higher, at least 1.9 times higher, at least 2 times higher, at least 3 times higher, at least 5 times higher, at least 10 times higher, up to at least 100 times higher or more when compared to a population of antibodies that have low levels of galactosylation.

In some embodiments, the population of antibodies that are highly galactosylated are highly fucosylated (have high levels of fucosylation). In some embodiments, the population of antibodies that are highly galactosylated and highly fucosylated has an increased level of complement dependent cytotoxicity (CDC) activity when compared to a population of antibodies that are low galactose and low fucose (have low levels of galactosylation and fucosylation). In some embodiments, the population of antibodies that is highly galactosylated and highly fucosylated and the population of antibodies that is low galactose and low fucose are directed to the same antigen epitope. In some embodiments, the population of antibodies that is highly galactosylated and highly fucosylated and the population of antibodies that is low galactose and low fucose are encoded by the same nucleic acid. In some embodiments, the nucleic acid encodes the antibody trastuzumab.

A population of antibodies that are low fucose or that have low levels of fucosylation, as used herein, refers to a population of antibodies wherein the level of fucosylation of the antibodies in the population is less than 50%, less than 40%, less than 30%, less than 20%, less than 10%, down to 0%.

In some embodiments, the CDC activity of a population of antibodies that is highly galactosylated and highly fucosylated is at least 1.1 times higher, at least 1.2 times higher, at least 1.3 times higher, at least 1.4 times higher, at least 1.5 times higher, at least 1.6 times higher, at least 1.7 times higher, at least 1.8 times higher, at least 1.9 times higher, at least 2 times higher, at least 3 times higher, at least 5 times higher, at least 10 times higher, up to at least 100 times higher or more when compared to a population of antibodies that is low galactose and low fucose.

In some embodiments, the population of antibodies that is highly galactosylated and is produced in mammary gland epithelial cells has an increased level of complement dependent cytotoxicity (CDC) activity when compared to a population of antibodies that is not produced in mammary gland epithelial cells. In some embodiments, the population of antibodies not produced in mammary gland epithelial cells is produced in cell culture. In some embodiments, the population of antibodies that is highly galactosylated produced in mammary gland epithelial cells and the population of antibodies that is not produced in mammary gland epithelial cells may be encoded by the same nucleic acid. In some embodiments, the nucleic acid encodes the antibody trastuzumab.

In some embodiments, the CDC activity of a population of antibodies that is highly galactosylated and is produced in mammary gland epithelial cells is at least 1.1 times higher, at least 1.2 times higher, at least 1.3 times higher, at least 1.4 times higher, at least 1.5 times higher, at least 1.6 times higher, at least 1.7 times higher, at least 1.8 times higher, at least 1.9 times higher, at least 2 times higher, at least 3 times higher, at least 5 times higher, at least 10 times higher, up to at least 100 times higher or more when compared to a population of antibodies that is not produced in mammary gland epithelial cells.

In one aspect, the compositions of the populations of antibodies disclosed herein have a high (complement dependent cytotoxicity) CDC activity. Antibodies can act as a therapeutic through various mechanisms, one of which is through CDC activity. Some therapeutic antibodies that bind to target cellular receptors can also bind proteins of the complement pathway. Binding of the complement proteins results in a complement cascade (through C1-complex activation) that eventually results in the formation of a “membrane attack complex” causing cell lysis and death of the cell to which the therapeutic antibody is bound (See e.g., Reff M. E. Blood 1994, 83: 435).

In some embodiments a population of antibodies that has an increased level of complement dependent cytotoxicity (CDC) activity, is a population of antibodies that induces a larger amount of cell lysis as compared to a population of antibodies that has does not have an increased level of complement dependent cytotoxicity (CDC) activity. Methods for determining the level of CDC are known in the art and are often based on determining the amount of cell lysis. Commercial kits for determining CDC activity can be purchased for instance from Genscript (Piscataway, N.J.).

### ADCC Activity

In one aspect, the population of anti-HER2 antibodies with high levels of galactosylation (e.g., at least 60%), has an increased level of antibody-dependent cellular cytotoxicity (ADCC) activity when compared to a population of antibodies that have low levels of galactosylation. In some embodiments, the disclosure provides compositions comprising populations of anti-HER2 antibodies with a high level of galactosylation wherein the population of antibodies is produced in mammary epithelial cells of a non-human mammal, and wherein the population of antibodies has an increased level of antibody-dependent cellular cytotoxicity (ADCC) activity when compared to a population of antibodies not produced in mammary gland epithelial cells. In some embodiments, the population of antibodies not produced in mammary gland epithelial cells is produced in cell culture.

In some embodiments, the population of antibodies that are highly galactosylated has an increased level of antibody-dependent cellular cytotoxicity (ADCC) when compared to a population of antibodies that are low galactose. In some embodiments, the ADCC activity of a population of antibodies that is highly galactosylated is at least 1.1 times higher, 1.2 times higher, 1.3 times higher, 1.4 times higher, 1.5 times higher, 1.6 times higher, 1.7 times higher, 1.8 times higher, 1.9 times higher, 2 times higher, 3 times higher, 5 times higher, 10 times higher, 100 times higher or more when compared to a population of antibodies that are low galactose.

In some embodiments, the population of antibodies that are highly galactosylated and is produced in mammary gland epithelial cells has an increased level of antibody-dependent cellular cytotoxicity (ADCC) when compared to a population of antibodies that is not produced in mammary gland epithelial cells. In some embodiments, the ADCC activity of a population of antibodies that is highly galactosylated and produced in mammary gland epithelial cells is at least 1.1 times higher, 1.2 times higher, 1.3 times higher, 1.4 times higher, 1.5 times higher, 1.6 times higher, 1.7 times higher, 1.8 times higher, 1.9 times higher, 2 times higher, 3 times higher, 5 times higher, 10 times higher, 100 times higher or more when compared to a population of antibodies that is not produced in mammary gland epithelial cells.

In some embodiments, the population of antibodies that is highly galactosylated and is produced in mammary gland epithelial cells has an increased level of antibody-dependent cellular cytotoxicity (ADCC) when compared to a population of antibodies that is not produced in mammary gland epithelial cells. In some embodiments, the population of antibodies not produced in mammary gland epithelial cells is produced in cell culture.

In one aspect, the compositions of the populations of antibodies disclosed herein have a high ADCC activity. Antibodies can act as a therapeutic through various mechanisms, one of which is through ADCC activity. Therapeutic antibodies that bind to cellular receptors on a target cell, and that include the Fc glycosylation site can also bind the Fc-receptor resulting in the anchoring of cells expressing the Fc-receptor to the target cell. The affinity of binding of the Fc regions of antibodies generally is dependent on the nature of the glycosylation of the Fc glycosylation site. The Fc receptor is found on a number of immune cells including natural killer cells, macrophages, neutrophils, and mast cells. Binding to the Fc receptor results in the immune cells inducing cytokines (such as IL-2) and phagocytosis to kill the target cell. In some embodiments, a population of antibodies that has an increased level of antibody-dependent cellular cytotoxicity (ADCC) activity is a population of antibodies that shows increased binding to cells expressing CD16 as compared to a population of antibodies that does not have an increased level of antibody-dependent cellular cytotoxicity (ADCC) activity. In some embodiments a population of antibodies that has an increased level of antibody-dependent cellular cytotoxicity (ADCC) activity is a population of antibodies that shows increased induction of IL-2 production (e.g., in natural killer cells) as compared to a population of antibodies that has does not have an increased level of antibody-dependent cellular cytotoxicity (ADCC) activity. Commercial kits for determining ADCC activity can be purchased for instance from Genscript (Piscataway, N.J.) and Promega (Madison, Wis.).

### Anti-HER2 Activity

In one aspect, the population of anti-HER2 antibodies with high levels of galactosylation (e.g., at least 60%) has an increased ability to suppress HER2 activity in a subject when compared to a population of antibodies that have low levels of galactosylation. In some embodiments, the disclosure provides compositions comprising populations of anti-HER2 antibodies with high levels of galactosylation, wherein the population of antibodies is produced in mammary epithelial cells of a non-human mammal, and wherein the population of antibodies has an increased ability to suppress HER2 activity in a subject when compared to a population of antibodies not produced in mammary gland epithelial cells.

In one aspect, the disclosure provides compositions comprising populations of anti-HER2 antibodies with high levels of galactosylation, wherein the population of antibodies is produced in mammary epithelial cells of a non-human mammal, and wherein the population of antibodies has an increased ability to bind HER2 when compared to a population of antibodies not produced in mammary gland epithelial cells.

In one aspect, the disclosure provides compositions comprising populations of anti-HER2 antibodies with high levels of galactosylation, wherein the population of antibodies is produced in mammary epithelial cells of a non-human mammal, and wherein the population of antibodies has an increased ability to suppress HER2 dimerization when compared to a population of antibodies not produced in mammary gland epithelial cells.

In some embodiments, the population of antibodies that are highly galactosylated has an increased ability to suppress HER2 activity, bind HER2 and/or suppress HER2 dimerization when compared to a population of antibodies that are low galactose. In some embodiments, the increased ability to suppress HER2 activity, bind HER2 and/or suppress HER2 dimerization of a population of antibodies that is highly galactosylated is at least 1.1 times higher, 1.2 times higher, 1.3 times higher, 1.4 times higher, 1.5 times higher, 1.6 times higher, 1.7 times higher, 1.8 times higher, 1.9 times higher, 2 times higher, 3 times higher, 5 times higher, 10 times higher, 100 times higher or more when compared to a population of antibodies that are low galactose.

In some embodiments, the population of antibodies that are highly galactosylated and is produced in mammary gland epithelial cells has an increased ability to suppress HER2 activity, bind HER2 and/or suppress HER2 dimerization when compared to a population of antibodies that is not produced in mammary gland epithelial cells. In some embodiments, the increased ability to suppress HER2 activity, bind HER2 and/or suppress HER2 dimerization of a population of antibodies that is highly galactosylated and produced in mammary gland epithelial cells is at least 1.1 times higher, 1.2 times higher, 1.3 times higher, 1.4 times higher, 1.5 times higher, 1.6 times higher, 1.7 times higher, 1.8 times higher, 1.9 times higher, 2 times higher, 3 times higher, 5 times higher, 10 times higher, 100 times higher or more when compared to a population of antibodies that is not produced in mammary gland epithelial cells.

In some embodiments, the population of antibodies that is highly galactosylated and is produced in mammary gland epithelial cells has increased ability to suppress HER2 activity, bind HER2 and/or suppress HER2 dimerization when compared to a population of antibodies that is not produced in mammary gland epithelial cells. In some embodiments, the population of antibodies not produced in mammary gland epithelial cells is produced in cell culture.

In some embodiments, the populations of anti-HER2 antibodies produced in mammary gland epithelial cells are superior to non-mammary gland epithelial cells produced antibodies in suppressing HER2 activity in a subject. Determining the level of HER2 activity in a subject can be evaluated for instance, by administering the population of antibodies to a subject suffering from a disease characterized by HER2 overexpression (e.g., HER2+ breast cancer). In some embodiments, the populations of anti-HER2 antibodies produced in mammary gland epithelial cells are superior to non-mammary gland epithelial cells produced antibodies in binding HER2. In some embodiments, the populations of anti-HER2 antibodies produced in mammary gland epithelial cells are superior to non-mammary gland epithelial cells produced antibodies in suppressing HER2 dimerization. Assays for determining the suppression of HER2 activity, the level of binding to HER2 and the dimerization of HER2 are well established (See e.g., Bookman et al., Journal of Clinical Oncology, Vol 21, No 2 (January 15), 2003: pp 283-290; Gee et al., Radiology. 2008 September; 248(3): 925-935; DeFazio-Eli et al., Breast Cancer Research 2011, 13:R44).

### Non-Human Mammary Gland Epithelial Cells and Transgenic Animals

In one aspect, the disclosure provides mammary gland epithelial cells that produce highly galactosylated anti-HER2 antibodies or populations of anti-HER2 antibodies with a high level of galactosylation.

In one aspect, the disclosure provides a transgenic non-human mammal that produces highly galactosylated anti-HER2 antibody or populations of anti-HER2 antibodies with a high level of galactosylation

In one aspect, the disclosure relates to mammalian mammary epithelial cells that produce glycosylated antibodies. Methods are provided herein for producing glycosylated antibodies in mammalian mammary epithelial cells. This can be accomplished in cell culture by culturing mammary epithelial cell (in vitro or ex vivo). This can also be accomplished in a transgenic animal (in vivo).

In some embodiments, the mammalian mammary gland epithelial cells are in a transgenic animal. In some embodiments, the mammalian mammary gland epithelial cells have been engineered to express recombinant antibodies in the milk of a transgenic animal, such as a mouse or goat. To accomplish this, the expression of the gene(s) encoding the recombinant protein can be, for example, under the control of the goat β-casein regulatory elements. Expression of recombinant proteins, e.g., antibodies, in both mice and goat milk has been established previously (see, e.g., US Patent Application US-2008-0118501-A1). In some embodiments, the expression is optimized for individual mammary duct epithelial cells that produce milk proteins.

Transgenic animals capable of producing recombinant antibodies can be generated according to methods known in the art (see, e.g., U.S. Pat. No. 5,945,577 and US Patent Application US-2008-0118501-A1). Animals suitable for transgenic expression, include, but are not limited to goat, sheep, bison, camel, cow, pig, rabbit, buffalo, horse, rat, mouse or llama. Suitable animals also include bovine, caprine, ovine and porcine, which relate to various species of cows, goats, sheep and pigs (or swine), respectively. Suitable animals also include ungulates. As used herein, “ungulate” is of or relating to a hoofed typically herbivorous quadruped mammal, including, without limitation, sheep, swine, goats, cattle and horses. Suitable animals also include dairy animals, such as goats and cattle, or mice. In some embodiments, the animal suitable for transgenic expression is a goat.

In one embodiment, transgenic animals are generated by generation of primary cells comprising a construct of interest followed by nuclear transfer of primary cell nucleus into enucleated oocytes. Primary cells comprising a construct of interest are produced by injecting or transfecting primary cells with a single construct comprising the coding sequence of an antibody of interest, e.g., the heavy and light chains of trastuzumab, or by co-transfecting or co-injecting primary cells with separate constructs comprising the coding sequences of the heavy and light chains of an antibody, e.g., trastuzumab. These cells are then expanded and characterized to assess transgene copy number, transgene structural integrity and chromosomal integration site. Cells with desired transgene copy number, transgene structural integrity and chromosomal integration site are then used for nuclear transfer to produce transgenic animals. As used herein, “nuclear transfer” refers to a method of cloning wherein the nucleus from a donor cell is transplanted into an enucleated oocyte.

Coding sequences for antibodies to be expressed in mammalian mammary epithelial cells can be obtained by screening libraries of genomic material or reverse-translated messenger RNA derived from the animal of choice (such as humans, cattle or mice), from sequence databases such as NCBI, Genbank, or by obtaining the sequences of antibodies using methods known in the art, e.g. peptide mapping. The sequences can be cloned into an appropriate plasmid vector and amplified in a suitable host organism, like E. coli. As used herein, a “vector” may be any of a number of nucleic acids into which a desired sequence may be inserted by restriction and ligation for transport between different genetic environments or for expression in a host cell. Vectors are typically composed of DNA although RNA vectors are also available. Vectors include, but are not limited to, plasmids and phagemids. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase or alkaline phosphatase), and genes which visibly affect the phenotype of transformed or transfected cells, hosts, colonies or plaques.

The coding sequence of antibodies or the heavy and light chains of antibodies of interest can be operatively linked to a control sequence which enables the coding sequence to be expressed in the milk of a transgenic non-human mammal. After amplification of the vector, the DNA construct can be excised, purified from the remains of the vector and introduced into expression vectors that can be used to produce transgenic animals. The transgenic animals will have the desired transgenic protein integrated into their genome.

A DNA sequence which is suitable for directing production to the milk of transgenic animals can carry a 5′-promoter region derived from a naturally-derived milk protein. This promoter is consequently under the control of hormonal and tissue-specific factors and is most active in lactating mammary tissue. In some embodiments the promoter used is a milk-specific promoter. As used herein, a “milk-specific promoter” is a promoter that naturally directs expression of a gene in a cell that secretes a protein into milk (e.g., a mammary epithelial cell) and includes, for example, the casein promoters, e.g., α-casein promoter (e.g., alpha S-1 casein promoter and alpha S2-casein promoter), β-casein promoter (e.g., the goat beta casein gene promoter (DiTullio, BIOTECHNOLOGY 10:74-77, 1992), γ-casein promoter, κ-casein promoter, whey acidic protein (WAP) promoter (Gorton et al., BIOTECHNOLOGY 5: 1183-1187, 1987), β-lactoglobulin promoter (Clark et al., BIOTECHNOLOGY 7: 487-492, 1989) and α-lactalbumin promoter (Soulier et al., FEBS LETTS. 297:13, 1992). Also included in this definition are promoters that are specifically activated in mammary tissue, such as, for example, the long terminal repeat (LTR) promoter of the mouse mammary tumor virus (MMTV). In some embodiments the promoter is a caprine beta casein promoter.

The promoter can be operably linked to a DNA sequence directing the production of a protein leader sequence which directs the secretion of the transgenic protein across the mammary epithelium into the milk. As used herein, a coding sequence and regulatory sequences (e.g., a promoter) are said to be “operably joined” or “operably linked” when they are linked in such a way as to place the expression or transcription of the coding sequence under the influence or control of the regulatory sequences. As used herein, a “leader sequence” or “signal sequence” is a nucleic acid sequence that encodes a protein secretory signal, and, when operably linked to a downstream nucleic acid molecule encoding a transgenic protein directs secretion. The leader sequence may be the native human leader sequence, an artificially-derived leader, or may be obtained from the same gene as the promoter used to direct transcription of the transgene coding sequence, or from another protein that is normally secreted from a cell, such as a mammalian mammary epithelial cell. In some embodiments a 3′-sequence, which can be derived from a naturally secreted milk protein, can be added to improve stability of mRNA.

In some embodiments, to produce primary cell lines containing a construct (e.g., encoding an trastuzumab antibody) for use in producing transgenic goats by nuclear transfer, the heavy and light chain constructs can be transfected into primary goat skin epithelial cells, which are expanded and fully characterized to assess transgene copy number, transgene structural integrity and chromosomal integration site. As used herein, “nuclear transfer” refers to a method of cloning wherein the nucleus from a donor cell is transplanted into an enucleated oocyte.

Cloning will result in a multiplicity of transgenic animals—each capable of producing an antibody or other gene construct of interest. The production methods include the use of the cloned animals and the offspring of those animals. Cloning also encompasses the nuclear transfer of fetuses, nuclear transfer, tissue and organ transplantation and the creation of chimeric offspring. One step of the cloning process comprises transferring the genome of a cell, e.g., a primary cell that contains the transgene of interest into an enucleated oocyte. As used herein, “transgene” refers to any piece of a nucleic acid molecule that is inserted by artifice into a cell, or an ancestor thereof, and becomes part of the genome of an animal which develops from that cell. Such a transgene may include a gene which is partly or entirely exogenous (i.e., foreign) to the transgenic animal, or may represent a gene having identity to an endogenous gene of the animal. Suitable mammalian sources for oocytes include goats, sheep, cows, pigs, rabbits, guinea pigs, mice, hamsters, rats, non-human primates, etc. Preferably, oocytes are obtained from ungulates, and most preferably goats or cattle. Methods for isolation of oocytes are well known in the art. Essentially, the process comprises isolating oocytes from the ovaries or reproductive tract of a mammal, e.g., a goat. A readily available source of ungulate oocytes is from hormonally-induced female animals. For the successful use of techniques such as genetic engineering, nuclear transfer and cloning, oocytes may preferably be matured in vivo before these cells may be used as recipient cells for nuclear transfer, and before they were fertilized by the sperm cell to develop into an embryo. Metaphase II stage oocytes, which have been matured in vivo, have been successfully used in nuclear transfer techniques. Essentially, mature metaphase II oocytes are collected surgically from either non-super ovulated or super ovulated animals several hours past the onset of estrus or past the injection of human chorionic gonadotropin (hCG) or similar hormone.

In some embodiments, the transgenic animals (e.g., goats) and mammary epithelial cells are generated through microinjection. Microinjection in goats is described for instance in U.S. Pat. No. 7,928,064. Briefly, fertilized goat eggs are collected from the PBS oviductal flushings on a stereomicroscope, and washed in medium containing 10% fetal bovine serum (FBS). In cases where the pronuclei were visible, the embryos can be immediately microinjected. If pronuclei are not visible, the embryos can be placed media for short term culture until the pronuclei became visible (Selgrath, et al., Theriogenology, 1990. p. 1195-1205). One-cell goat embryos are placed in a microdrop of medium under oil on a glass depression slide. Fertilized eggs having two visible pronuclei and can be immobilized on a flame-polished holding micropipet on an upright microscope with a fixed stage. A pronucleus can be microinjected with the appropriate antibody encoding construct in injection buffer using a fine glass microneedle (Selgrath, et al., Theriogenology, 1990. p. 1195-1205). After microinjection, surviving embryos are placed in a culture and incubated until the recipient animals are prepared for embryo transfer (Selgrath, et al., Theriogenology, 1990. p. 1195-1205).

Thus, in one aspect the disclosure provides mammary gland epithelial cells that produce the antibodies or populations of antibodies disclosed herein. In some embodiments, the antibody comprises a nucleic acid comprising SEQ ID NO:3 and a nucleic acid comprising SEQ ID NO:4. In some embodiments, the nucleic acid comprising SEQ ID NO:3 and the nucleic acid comprising SEQ ID NO:4 are connected. “Connected” is used herein to mean the nucleic acids are physically linked, e.g., within the same vector or within approximately the same genomic location. In some embodiments, the mammary epithelial cells above are in a transgenic non-human mammal. In some embodiments, the transgenic non-human mammal is a goat.

A nucleic acid sequence encoding the heavy chain of trastuzumab is provided in SEQ ID NO:3:

ATGGAGTTCGGCCTGAGCTGGCTGTTCCTGGTGGCCATCCTGAAGGGCGTG
CAGTGCGAGGTGCAGCTGGTCGAGAGCGGAGGAGGACTGGTCCAGCCTGGC
GGCAGCCTGAGACTGAGCTGCGCCGCCAGCGGCTTCAACATCAAGGACACC
TACATCCACTGGGTGCGCCAGGCTCCAGGGAAAGGGCTCGAATGGGTGGCC
AGGATCTACCCCACCAACGGCTACACCAGATACGCCGACAGCGTGAAGGGC
AGGTTCACCATCAGCGCCGACACCAGCAAGAACACCGCCTACCTGCAGATG
AACAGCCTGAGGGCCGAGGACACCGCCGTGTACTACTGCAGCAGATGGGGT
GGGGATGGCTTCTACGCCATGGACTACTGGGGGCAGGGCACACTGGTCACA
GTCTCCAGCGCCAGCACCAAGGGCCCCAGCGTGTTCCCCCTGGCTCCTTCC
TCTAAATCCACAAGCGGCGGCACCGCTGCCCTGGGCTGCCTGGTGAAGGAC
TACTTCCCCGAGCCCGTGACCGTGTCTTGGAACTCTGGCGCCCTGACCTCC
GGCGTGCACACCTTCCCCGCCGTGCTGCAGAGCAGCGGCCTGTACAGCCTG
AGCAGCGTGGTGACCGTGCCCTCTTCCTCTCTCGGAACACAGACCTACATC
TGCAACGTGAACCACAAGCCCAGCAACACCAAGGTGGACAAGAAGGTGGAG
CCCAAGAGCTGCGACAAGACCCATACATGTCCTCCCTGTCCTGCTCCTGAG
CTGCTGGGCGGACCCTCCGTGTTCCTGTTCCCCCCCAAGCCCAAGGACACC
CTGATGATCAGCAGGACCCCCGAGGTGACCTGCGTGGTGGTGGACGTGTCC
CACGAGGACCCTGAGGTGAAGTTCAACTGGTACGTGGACGGCGTGGAGGTG
CACAACGCCAAGACCAAGCCCAGAGAGGAGCAGTACAACAGCACCTACAGG
GTGGTGTCCGTGCTGACCGTGCTGCACCAGGACTGGCTGAACGGCAAAGAA
TACAAGTGCAAAGTCTCCAACAAGGCCCTGCCAGCCCCCATCGAAAAGACC
ATCAGCAAGGCCAAGGGCCAGCCTCGCGAGCCCCAGGTGTACACCCTGCCC
CCCTCCCGCGACGAGCTGACCAAGAACCAGGTGTCCCTGACCTGTCTGGTG
AAGGGCTTCTACCCCAGCGATATCGCCGTGGAGTGGGAGAGCAACGGCCAG
CCCGAGAACAACTACAAGACCACCCCCCCTGTGCTGGACAGCGACGGCAGC
TTCTTCCTGTACAGCAAGCTGACCGTGGACAAGAGCAGGTGGCAGCAGGGA
AATGTCTTTTCCTGTTCCGTCATGCATGAAGCTCTGCACAACCACTACACC
CAGAAGTCCCTGAGCCTGAGCCCCGGCAAGTGATAG

A nucleic acid sequence encoding the light chain of trastuzumab is provided in SEQ ID NO:4:

ATGGACATGAGAGTGCCTGCCCAGCTCCTGGGACTCCTCCTCCTGTGGCTC
AGGGGTGCTCGCTGCGATATCCAGATGACTCAGTCTCCTTCTTCCCTCTCC
GCCAGCGTGGGCGACAGAGTGACCATCACCTGCAGGGCCAGCCAGGACGTG
AACACCGCCGTGGCCTGGTATCAGCAGAAGCCCGGCAAGGCCCCCAAGCTG
CTGATCTACAGCGCCAGCTTCCTGTACAGCGGCGTGCCCAGCAGGTTCAGC
GGCAGCAGAAGCGGCACCGACTTCACCCTGACCATCAGCAGCCTGCAGCCC
GAGGACTTCGCCACCTACTACTGCCAGCAGCACTACACCACCCCCCCCACC
TTCGGCCAGGGCACCAAGGTGGAGATCAAGAGGACCGTGGCCGCTCCCAGC
GTGTTCATCTTCCCCCCCAGCGACGAGCAGCTGAAGTCCGGCACCGCCTCC
GTGGTGTGCCTGCTGAACAACTTCTACCCCCGCGAGGCCAAGGTGCAGTGG
AAGGTGGACAACGCCCTGCAGAGCGGCAACAGCCAGGAGAGCGTCACCGAG
CAGGACAGCAAGGACTCCACCTACAGCCTGAGCAGCACCCTGACCCTGAGC
AAGGCCGACTACGAGAAGCACAAGGTGTACGCCTGCGAGGTGACCCACCAG
GGCCTGTCCAGCCCCGTGACCAAGAGCTTCAACAGGGGCGAGTGCTGA

In another aspect the disclosure provides a method for the production of a transgenic antibody, and populations thereof, the process comprising expressing in the milk of a transgenic non-human mammal a transgenic antibody encoded by a nucleic acid construct. In some embodiments, the method for producing the antibodies of the disclosure comprises:

(a) transfecting non-human mammalian cells with a transgene DNA construct encoding an anti-HER2 antibody;

(b) selecting cells in which said anti-HER2 transgene DNA construct has been inserted into the genome of the cells; and

(c) performing a first nuclear transfer procedure to generate a non-human transgenic mammal heterozygous for the anti-HER2 antibody and that can express it in its milk.

In some embodiments, the anti-HER2 antibody is trastuzumab.

In some embodiments, the transgene DNA construct comprises SEQ ID NO:3 and/or SEQ ID NO:4. In some embodiments, the non-human transgenic mammal is a goat.

In another aspect, the disclosure provides a method of:

(a) providing a non-human transgenic mammal engineered to express an anti-HER2 antibody,

(b) expressing the anti-HER2 antibody in the milk of the non-human transgenic mammal; and

(c) isolating the anti-HER2 antibody expressed in the milk.

In some embodiments, the anti-HER2 antibody comprises a heavy chain comprising SEQ ID NO:1 and a light chain comprising SEQ ID NO:2. In some embodiments, the anti-HER2 antibody is trastuzumab.

One of the tools used to predict the quantity and quality of the recombinant protein expressed in the mammary gland is through the induction of lactation (Ebert KM, 1994). Induced lactation allows for the expression and analysis of protein from the early stage of transgenic production rather than from the first natural lactation resulting from pregnancy, which is at least a year later. Induction of lactation can be done either hormonally or manually.

In some embodiments, the compositions of glycosylated antibodies provided herein further comprise milk. In some embodiments, the methods provided herein include a step of isolating a population of antibodies from the milk of a transgenic animal. Methods for isolating antibodies from the milk of transgenic animal are known in the art and are described for instance in Pollock et al., Journal of Immunological Methods, Volume 231, Issues 1-2, 10 Dec. 1999, Pages 147-157. In some embodiments, the methods provided herein include a step of purifying glycosylated antibodies with a desired amount of galactosylation.

### Methods of Treatment, Pharmaceutical Compositions, Dosage, and Administration

In one aspect, the disclosure provides methods comprising administering highly galactosylated anti-HER2 antibodies, compositions of highly galactosylated anti-HER2 antibodies, populations of antibodies with a high level of galactosylated anti-HER2 antibodies or compositions comprising populations of antibodies with a high level of galactosylated anti-HER2 antibodies, to a subject in need thereof. In some embodiments, the galactosylated anti-HER2 antibody is trastuzumab. In some embodiment, the subject has cancer. In some embodiments, the subject has cancer characterized by overexpression of HER2 (HER2+ cancer). Methods for determining the HER2 status of a cancer are routing in the art, for instance, two-FDA approved commercial kits are available (HercepTest™ by Dako, and Pathway Her-2 by Ventana). In some embodiments, the HER2+ cancer is breast, ovarian, stomach or uterine cancer.

In one aspect, the disclosure provides methods comprising administering highly galactosylated anti-HER2 antibodies, compositions of highly galactosylated anti-HER2 antibodies, populations of antibodies with a high level of galactosylated anti-HER2 antibodies or compositions comprising populations of antibodies with a high level of galactosylated anti-HER2 antibodies, to a subject in need thereof. In some embodiments, the galactosylated anti-HER2 antibody is trastuzumab. In some embodiment, the subject has breast cancer, biliary tract cancer; bladder cancer; brain cancer including glioblastomas and medulloblastomas; cervical cancer; choriocarcinoma; colon cancer; endometrial cancer; esophageal cancer; gastric cancer; hematological neoplasms including acute lymphocytic and myelogenous leukemia; T-cell acute lymphoblastic leukemia/lymphoma; hairy cell leukemia; chronic myelogenous leukemia, multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma or Wilms tumor.

In one aspect, the disclosure provides methods comprising administering highly galactosylated anti-HER2 antibodies, compositions of highly galactosylated anti-HER2 antibodies, populations of antibodies with a high level of galactosylated anti-HER2 antibodies or compositions comprising populations of antibodies with a high level of galactosylated anti-HER2 antibodies, to a subject in need thereof. In some embodiments, the galactosylated anti-HER2 antibody is trastuzumab. In some embodiments, the methods further include the administration of a chemotherapeutic agent in addition to the anti-HER2 antibody. Chemotherapeutic reagents include methotrexate, vincristine, adriamycin, cisplatin, non-sugar containing chloroethylnitrosoureas, 5-fluorouracil, mitomycin C, bleomycin, doxorubicin, dacarbazine, taxol, fragyline, Meglamine GLA, valrubicin, carmustaine and poliferposan, MMI270, BAY 12-9566, RAS famesyl transferase inhibitor, famesyl transferase inhibitor, MMP, MTA/LY231514, LY264618/Lometexol, Glamolec, CI-994, TNP-470, Hycamtin/Topotecan, PKC412, Valspodar/PSC833, Novantrone/Mitroxantrone, Metaret/Suramin, Batimastat, E7070, BCH-4556, CS-682, 9-AC, AG3340, AG3433, Incel/VX-710, VX-853, ZD0101, IS1641, ODN 698, TA 2516/Marmistat, BB2516/Marmistat, CDP 845, D2163, PD183805, DX8951f, Lemonal DP 2202, FK 317, Picibanil/OK-432, AD 32/Valrubicin, Metastron/strontium derivative, Temodal/Temozolomide, Evacet/liposomal doxorubicin, Yewtaxan/Paclitaxel, Taxol/Paclitaxel, Xeload/Capecitabine, Furtulon/Doxifluridine, Cyclopax/oral paclitaxel, Oral Taxoid, SPU-077/Cisplatin, HMR 1275/Flavopiridol, CP-358 (774)/EGFR, CP-609 (754)/RAS oncogene inhibitor, BMS-182751/oral platinum, UFT(Tegafur/Uracil), Ergamisol/Levamisole, Eniluracil/776C85/5FU enhancer, Campto/Levamisole, Camptosar/Irinotecan, Tumodex/Ralitrexed, Leustatin/Cladribine, Paxex/Paclitaxel, Doxil/liposomal doxorubicin, Caelyx/liposomal doxorubicin, Fludara/Fludarabine, Pharmarubicin/Epirubicin, DepoCyt, ZD1839, LU 79553/Bis-Naphtalimide, LU 103793/Dolastain, Caetyx/liposomal doxorubicin, Gemzar/Gemcitabine, ZD 0473/Anormed, YM 116, iodine seeds, CDK4 and CDK2 inhibitors, PARP inhibitors, D4809/Dexifosamide, Ifes/Mesnex/Ifosamide, Vumon/Teniposide, Paraplatin/Carboplatin, Plantinol/cisplatin, Vepeside/Etoposide, ZD 9331, Taxotere/Docetaxel, prodrug of guanine arabinoside, Taxane Analog, nitrosoureas, alkylating agents such as melphelan and cyclophosphamide, Aminoglutethimide, Asparaginase, Busulfan, Carboplatin, Chlorombucil, Cytarabine HCl, Dactinomycin, Daunorubicin HCl, Estramustine phosphate sodium, Etoposide (VP16-213), Floxuridine, Fluorouracil (5-FU), Flutamide, Hydroxyurea (hydroxycarbamide), Ifosfamide, Interferon Alfa-2a, Alfa-2b, Leuprolide acetate (LHRH-releasing factor analogue), Lomustine (CCNU), Mechlorethamine HCl (nitrogen mustard), Mercaptopurine, Mesna, Mitotane (o.p′-DDD), Mitoxantrone HCl, Octreotide, Plicamycin, Procarbazine HCl, Streptozocin, Tamoxifen citrate, Thioguanine, Thiotepa, Vinblastine sulfate, Amsacrine (m-AMSA), Azacitidine, Erthropoietin, Hexamethylmelamine (HMM), Interleukin 2, Mitoguazone (methyl-GAG; methyl glyoxal bis-guanylhydrazone; MGBG), Pentostatin (2′deoxycoformycin), Semustine (methyl-CCNU), Teniposide (VM-26) and Vindesine sulfate, but are not so limited.

In one aspect, the disclosure provides pharmaceutical compositions which comprise an amount of an antibody or population of antibodies and a pharmaceutically acceptable vehicle, diluent or carrier. In some embodiments, the compositions comprise milk.

In one aspect, the disclosure provides a method of treating a subject, comprising administering to a subject a composition provided in an amount effective to treat a disease the subject has or is at risk of having is provided. In one embodiment the subject is a human. In another embodiment the subject is a non-human animal, e.g., a dog, cat, horse, cow, pig, sheep, goat or primate.

According to embodiments that involve administering to a subject in need of treatment a therapeutically effective amount of the antibodies as provided herein, “therapeutically effective” or “an amount effective to treat” denotes the amount of antibody or of a composition needed to inhibit or reverse a disease condition (e.g., to treat cancer). Determining a therapeutically effective amount specifically depends on such factors as toxicity and efficacy of the medicament. These factors will differ depending on other factors such as potency, relative bioavailability, patient body weight, severity of adverse side-effects and preferred mode of administration. Toxicity may be determined using methods well known in the art. Efficacy may be determined utilizing the same guidance. Efficacy, for example, can be measured by a decrease in the progress of the cancer. A pharmaceutically effective amount, therefore, is an amount that is deemed by the clinician to be toxicologically tolerable, yet efficacious.

Dosage may be adjusted appropriately to achieve desired drug (e.g., anti-HER2 antibodies) levels, local or systemic, depending upon the mode of administration. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of antibodies. Appropriate systemic levels can be determined by, for example, measurement of the patient's peak or sustained plasma level of the drug. “Dose” and “dosage” are used interchangeably herein.

In some embodiments, the amount of antibody or pharmaceutical composition administered to a subject is 50 to 500 mg/kg, 100 to 400 mg/kg, or 200 to 300 mg/kg per week. In one embodiment the amount of antibody or pharmaceutical composition administered to a subject is 250 mg/kg per week. In some embodiments, an initial dose of 400 mg/kg is administered a subject the first week, followed by administration of 250 mg/kg to the subject in subsequent weeks. In some embodiments the administration rate is less than 10 mg/min. In some embodiments, administration of the antibody or pharmaceutical composition to a subject occurs at least one hour prior to treatment with another therapeutic agent. In some embodiments, a pre-treatment is administered prior to administration of the antibody or pharmaceutical composition.

In some embodiments the compositions provided are employed for in vivo applications. Depending on the intended mode of administration in vivo the compositions used may be in the dosage form of solid, semi-solid or liquid such as, e.g., tablets, pills, powders, capsules, gels, ointments, liquids, suspensions, or the like. Preferably, the compositions are administered in unit dosage forms suitable for single administration of precise dosage amounts. The compositions may also include, depending on the formulation desired, pharmaceutically acceptable carriers or diluents, which are defined as aqueous-based vehicles commonly used to formulate pharmaceutical compositions for animal or human administration. The diluent is selected so as not to affect the biological activity of the human recombinant protein of interest. Examples of such diluents are distilled water, physiological saline, Ringer's solution, dextrose solution, and Hank's solution. The same diluents may be used to reconstitute a lyophilized recombinant protein of interest. In addition, the pharmaceutical composition may also include other medicinal agents, pharmaceutical agents, carriers, adjuvants, nontoxic, non-therapeutic, non-immunogenic stabilizers, etc. Effective amounts of such diluent or carrier are amounts which are effective to obtain a pharmaceutically acceptable formulation in terms of solubility of components, biological activity, etc. In some embodiments the compositions provided herein are sterile.

Administration during in vivo treatment may be by any number of routes, including oral, parenteral, intramuscular, intranasal, sublingual, intratracheal, inhalation, ocular, vaginal, and rectal. Intracapsular, intravenous, and intraperitoneal routes of administration may also be employed. The skilled artisan recognizes that the route of administration varies depending on the disorder to be treated. For example, the compositions or antibodies herein may be administered to a subject via oral, parenteral or topical administration. In one embodiment, the compositions or antibodies herein are administered by intravenous infusion.

The compositions, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compositions in water soluble form. Additionally, suspensions of the active compositions may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compositions to allow for the preparation of highly concentrated solutions. Alternatively, the active compositions may be in powder form for constitution with a suitable vehicle, e.g., sterile pyrogen-free water, before use.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. The component or components may be chemically modified so that oral delivery of the antibodies is efficacious. Generally, the chemical modification contemplated is the attachment of at least one molecule to the antibodies, where said molecule permits (a) inhibition of proteolysis; and (b) uptake into the blood stream from the stomach or intestine. Also desired is the increase in overall stability of the antibodies and increase in circulation time in the body. Examples of such molecules include: polyethylene glycol, copolymers of ethylene glycol and propylene glycol, carboxymethyl cellulose, dextran, polyvinyl alcohol, polyvinyl pyrrolidone and polyproline. Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189. Other polymers that could be used are poly-1,3-dioxolane and poly-1,3,6-tioxocane. Preferred for pharmaceutical usage, as indicated above, are polyethylene glycol molecules. For oral compositions, the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the antibody or by release of the biologically active material beyond the stomach environment, such as in the intestine.

For buccal administration, the compositions may take the form of tablets or lozenges formulated in conventional manner.

For administration by inhalation, the compositions for use according to the present disclosure may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compositions and a suitable powder base such as lactose or starch.

Also contemplated herein is pulmonary delivery. The compositions can be delivered to the lungs of a mammal while inhaling and traverses across the lung epithelial lining to the blood stream. Contemplated for use in the practice of this disclosure are a wide range of mechanical devices designed for pulmonary delivery of therapeutic products, including but not limited to nebulizers, metered dose inhalers, and powder inhalers, all of which are familiar to those skilled in the art.

Nasal delivery of a pharmaceutical composition disclosed herein is also contemplated. Nasal delivery allows the passage of a pharmaceutical composition of the present disclosure to the blood stream directly after administering the therapeutic product to the nose, without the necessity for deposition of the product in the lung. Formulations for nasal delivery include those with dextran or cyclodextran.

The compositions may also be formulated in rectal or vaginal compositions such as suppositories or retention enemas, e.g., containing conventional suppository bases such as cocoa butter or other glycerides.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compositions, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The antibodies and optionally other therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions of the disclosure contain an effective amount of the antibodies and optionally therapeutic agents included in a pharmaceutically-acceptable carrier. The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compositions of the present disclosure, and with each other, in a manner such that there is no interaction which would substantially impair the desired pharmaceutical efficiency.

The therapeutic agent(s), including specifically but not limited to the antibodies, may be provided in particles. Particles as used herein means nano or microparticles (or in some instances larger) which can consist in whole or in part of the antibody or other therapeutic agents administered with the antibody. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the antibody in a solution or in a semi-solid state. The particles may be of virtually any shape.

### Methods of Production of Antibodies

In one aspect, the disclosure provides methods for production of highly galactosylated anti-HER2 antibodies and populations with high levels of galactosylated antibodies.

In one aspect, the disclosure provides a method for producing a population of antibodies, comprising: expressing the population of antibodies in mammary gland epithelial cells of a non-human mammal such that a population of antibodies is produced, wherein the antibody is an anti-HER2 antibody, and wherein the level of galactosylation of the antibodies in the population is at least 70%. In some embodiments, the anti-HER2 antibody is trastuzumab. In some embodiments, the mammary gland epithelial cells are in culture and are transfected with a nucleic acid that comprises a sequence that encodes the antibody. In some embodiments, the nucleic acid comprise SEQ ID NO:3 and SEQ ID NO:4. In some embodiments, the mammary gland epithelial cells are in a non-human mammal engineered to express a nucleic acid that comprises a sequence that encodes the antibody in its mammary gland. In some embodiments, the mammary gland epithelial cells are goat, sheep, bison, camel, cow, pig, rabbit, buffalo, horse, rat, mouse or llama mammary gland epithelial cells. In some embodiments, the mammary gland epithelial cells are goat mammary gland epithelial cells.

In one aspect the disclosure provides mammary gland epithelial cells that express the highly galactosylated anti-HER2 antibodies or populations with high levels of galactosylated antibodies disclosed herein.

In one aspect the disclosure provides a transgenic non-human mammal comprising mammary gland epithelial cells that express the highly galactosylated anti-HER2 antibodies or populations with high levels of galactosylated antibodies disclosed herein.

In one aspect the disclosure provides a method for the production of a glycosylated antibody or population of glycosylated antibodies, the process comprising expressing in the milk of a transgenic non-human mammal a glycosylated antibody encoded by a nucleic acid construct. In one embodiment the mammalian mammary epithelial cells are of a non-human mammal engineered to express the antibody in its milk. In yet another embodiment the mammalian mammary epithelial cells are mammalian mammary epithelial cells in culture.

In another embodiment the method comprises:

(a) providing a non-human transgenic mammal engineered to express an antibody,

(b) expressing the antibody in the milk of the non-human transgenic mammal;

(c) isolating the antibodies expressed in the milk; and

(d) detecting the presence galactose on the isolated antibodies.

In yet another embodiment the method, comprises: producing a population of glycosylated antibodies in mammary gland epithelial cells such that the population of glycosylated antibodies produced comprises a specific percentage of galactosylation (e.g., at least 70%, at least 80%, at least 90%, or higher). In some embodiment, the antibody is an anti-HER2 antibody. In some embodiments, the glycosylated antibodies comprise a heavy chain comprising SEQ ID NO; 1 and a light chain comprising SEQ ID NO:2. In some embodiments, this method is performed in vitro. In other embodiments, this method is performed in vivo, e.g., in the mammary gland of a transgenic goat.

In some embodiments the methods above further comprise steps for inducing lactation. In still other embodiments the methods further comprise additional isolation and/or purification steps. In yet other embodiments the methods further comprise steps for comparing the glycosylation pattern of the antibodies obtained with antibodies produced in cell culture, e.g. non-mammary cell culture. In further embodiments, the methods further comprise steps for comparing the glycosylation pattern of the antibodies obtained to antibodies produced by non-mammary epithelial cells. Such cells can be cells of a cell culture. In some embodiments, the glycosylation pattern is the amount of galactose present on an antibody or population of antibodies. In some embodiments, the method further comprises comparing the percentage of galactosylation present in the population of glycosylated antibodies to the percentage of galactosylation present in a population of glycosylated antibodies produced in cell culture, e.g. non-mammary cell culture. Experimental techniques for assessing the glycosylation pattern of the antibodies can be any of those known to those of ordinary skill in the art or as provided herein, such as below in the Examples. Such methods include, e.g., liquid chromatography mass spectrometry, tandem mass spectrometry, and Western blot analysis.

The antibodies can be obtained, in some embodiments, by collecting the antibodies from the milk of a transgenic animal produced as provided herein or from an offspring of said transgenic animal. In some embodiments the antibodies produced by the transgenic mammal is produced at a level of at least 1 gram per liter of milk produced. Advantageously, the method according to the invention allows production of at least 4 grams per liter of milk. In some embodiments, methods described herein allow for production of at least 5, 10, 15, 20, 25, 30, 35, 40, 45, 50, 55, 60, 65 or 70 grams per liter. In some embodiments, methods described herein can allow for production of at least 60 grams per liter. In some embodiments, methods described herein can allow for production of at least 70 grams per liter.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The methods and techniques of the present disclosure are generally performed according to conventional methods well-known in the art. Generally, nomenclatures used in connection with, and techniques of biochemistry, enzymology, molecular and cellular biology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated.

The present invention is further illustrated by the following Examples, which in no way should be construed as further limiting. The entire contents of all of the references (including literature references, issued patents, published patent applications, and co pending patent applications) cited throughout this application are hereby expressly incorporated by reference.

### EXAMPLES

### Materials and Methods

Generation of Transgenic Goats that Produce Trastuzumab

Transgenic goats were generated that include the nucleic acid sequence encoding the trastuzumab antibody in their genome. The goats producing trastuzumab were generated using traditional microinjection techniques (See e.g., U.S. Pat. No. 7,928,064). The cDNA encoding the heavy and light chain (SEQ ID NO:3 and SEQ ID NO:4) were ligated with the beta casein expression vector to yield constructs BC2601 HC and BC2602 LC. In these plasmids, the nucleic acid sequence encoding trastuzumab is under the control of a promoter facilitating the expression of trastuzumab in the mammary gland of the goats. The prokaryotic sequences were removed and the DNA microinjected into pre-implantation embryos of the goat. These embryos were then transferred to pseudo pregnant females. The progeny that resulted were screened for the presence of the transgenes. Those that carried both chains were identified as transgenic founders.

When age appropriate, the founder animals were bred. Following pregnancy and parturition they were milked. The time course was in days starting lactation after parturation (e.g., day 7,). The trastuzumab antibody was purified from the milk at each time point and characterized as described herein.

### Example 1

### Transgenically Produced Trastuzumab

The glycosylation pattern of the trastuzumab antibodies produced in the milk of transgenic goats was determined by releasing the N-glycans from antibody and running the released oligosaccharides on a column (“oligosaccharide signature”).

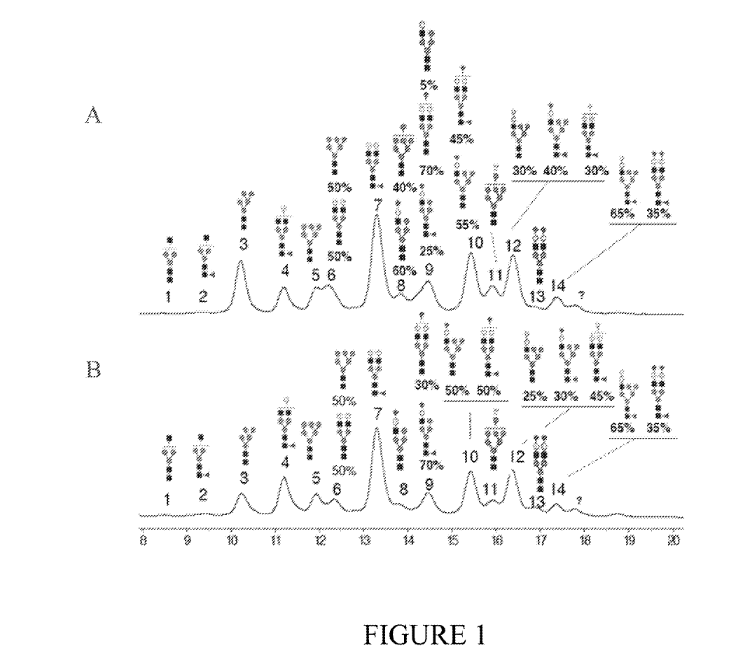

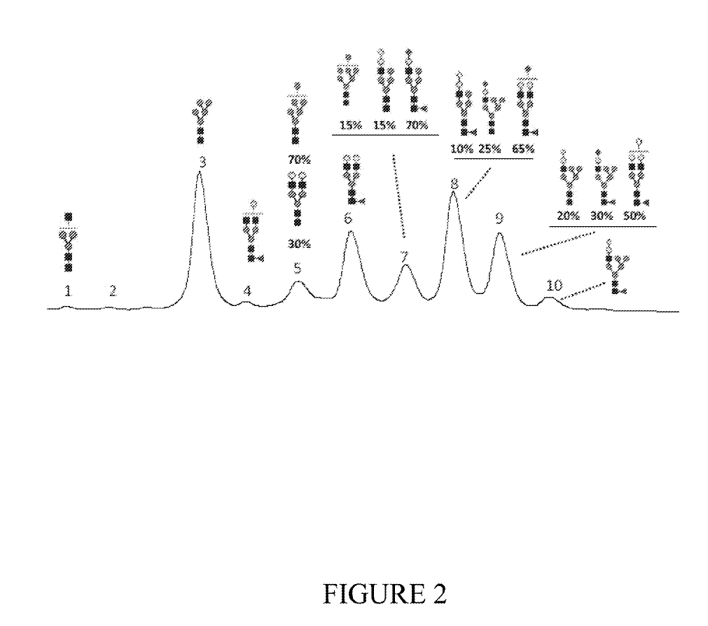

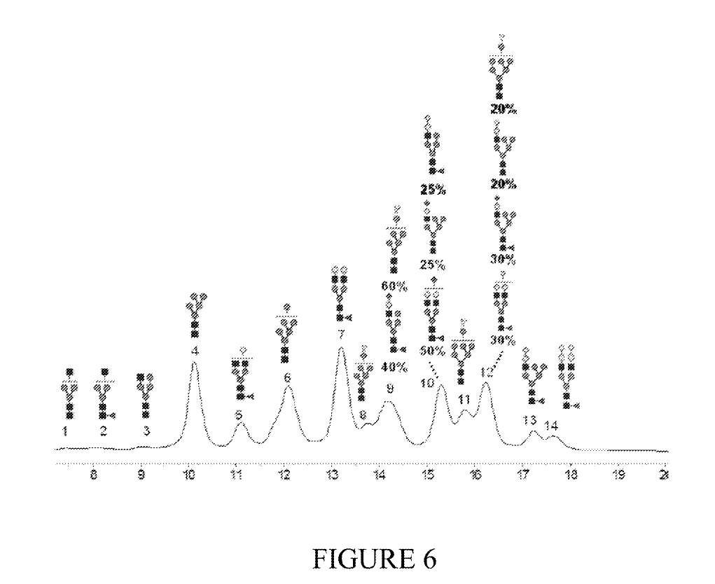

FIGS. 1-4 and 6 show the N-glycan oligosaccharides released from the transgenically produced trastuzumab antibody from goat #1 (FIGS. 2-4) and goat #2 (FIGS. 1 and 6). The monosaccharide groups are depicted as follows:

Black square: N-acetylGlucosamine (GlcNac)

Triangle: Fucose

Grey Circle: Mannose

White Circle: Galactose

Grey Diamond: N-GlycolylNeuraminic Acid (NGNA): a sialic acid

White Diamond: N-AcetylNeuraminic Acid (NANA): a sialic acid

FIG. 1 shows representative chromatograms of N-glycan oligosaccharides released from the transgenic trastuzumab antibody produced in the milk of goat #2. FIG. 1 shows that of the major N-glycan oligosaccharides produced (21 in FIG. 1A, and 20 in FIG. 1B), fourteen have at least one galactose in the N-glycan chain, with seven oligosaccharides having two galactoses. Six of the oligosaccharides are purely oligomannose. FIG. 1 also shows that of the major oligosaccharides produced, nine are fucosylated

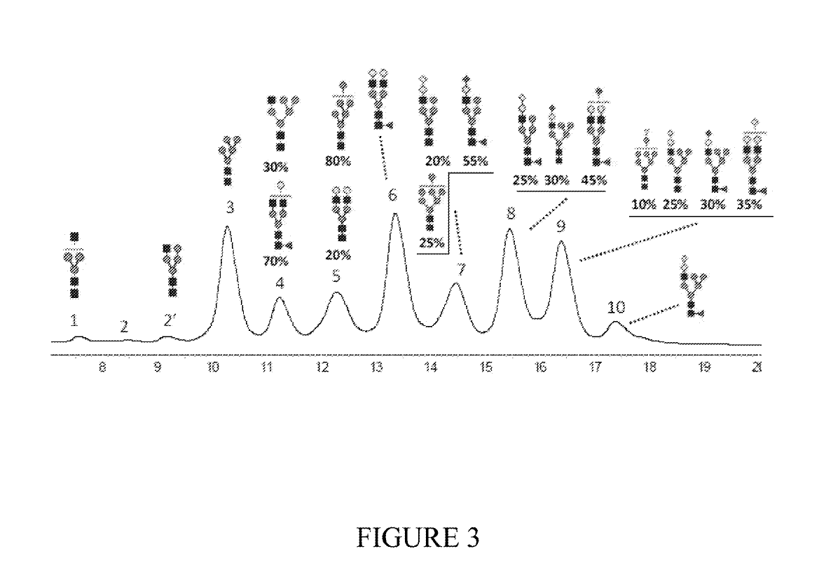

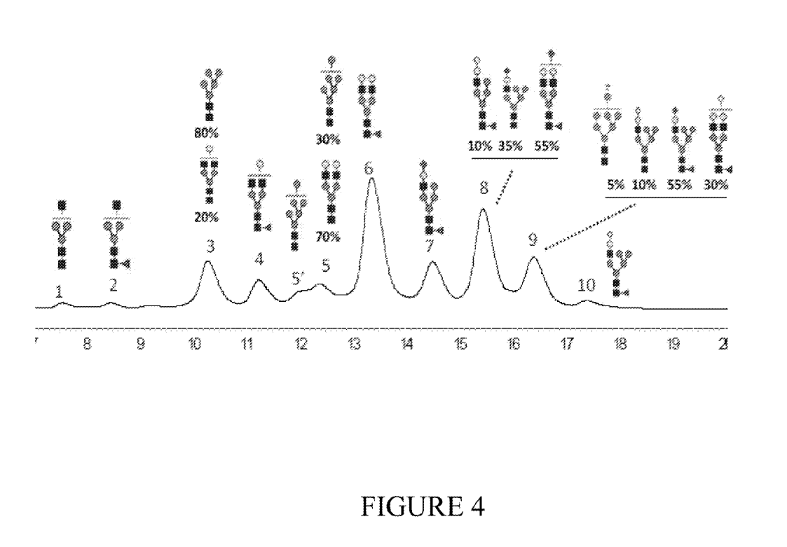

FIGS. 2-4 show chromatograms of N-glycan oligosaccharides released from the transgenically produced trastuzumab antibody in the milk of goat #1 as harvested after 7 days of lactation (FIG. 2), 15 days of lactation (FIG. 3), and 30 days of lactation (FIG. 4).

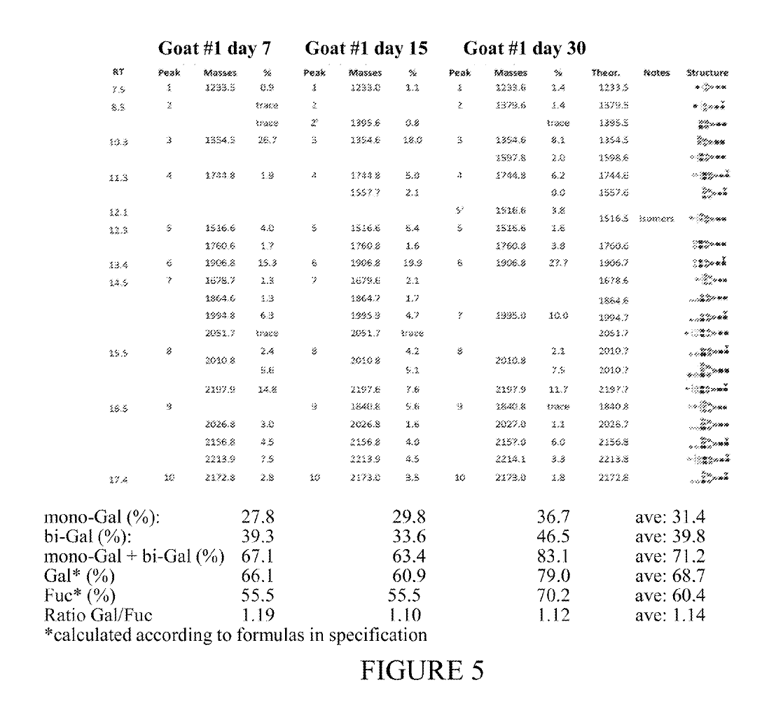

The relative percentages of all N-glycan oligosaccharides isolated from the trastuzumab antibody produced in the milk of goat #1 are depicted in FIG. 5. FIG. 5 also tabulates the overall percentage of mono-galactosylation, percentage of bi-galactosylation, percentage of total galactosylation (mono-galactosylation+bi-galactosylation), percentage of galactosylation as calculated according to the formula provided above, percentage of fucosylation as calculated according to the formula provided above, and the ratio of galactosylation to fucosylation of trastuzumab antibodies produced in goat #1. The results are also summarized in Table 1 below:

TABLE 1
N-glycan oligosaccharides isolated from
trastuzumab antibodies from goat #1

| | day 7 | day 15 | day 30 | average |
|---|---|---|---|---|
| mono-Gal (%): | 27.8 | 29.8 | 36.7 | 31.4 |
| bi-Gal (%): | 39.3 | 33.6 | 46.5 | 39.8 |
| mono-Gal + bi-Gal (%) | 67.1 | 63.4 | 83.1 | 71.2 |
| Gal* (%) | 66.1 | 60.9 | 79.0 | 68.7 |
| Fuc* (%) | 55.5 | 55.5 | 70.2 | 60.4 |
| Ratio Gal/Fuc | 1.19 | 1.10 | 1.12 | 1.14 |

*calculated according to formulas in specification

FIG. 6 shows a chromatogram of N-glycan oligosaccharides released from the transgenically produced trastuzumab antibody in the milk of goat #2 as harvested at day 7 of lactation.

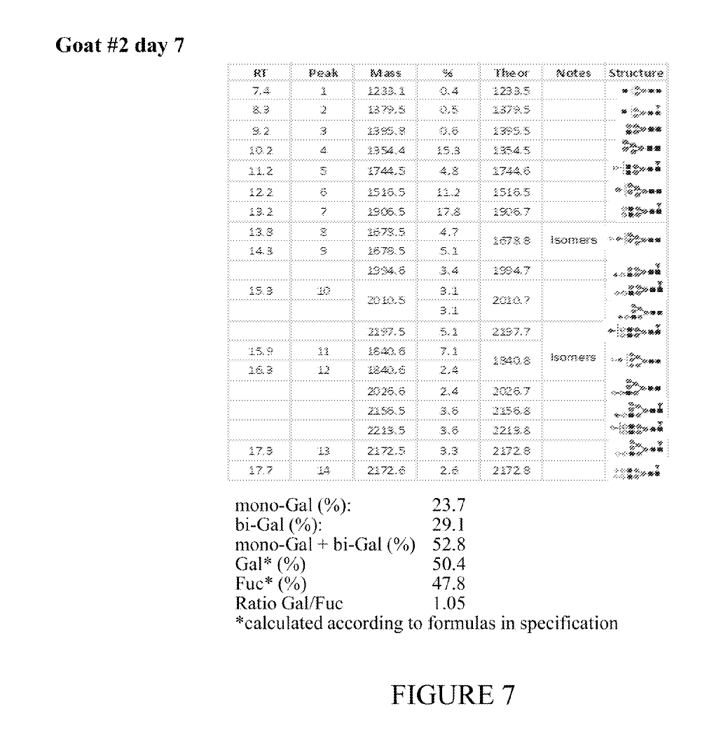

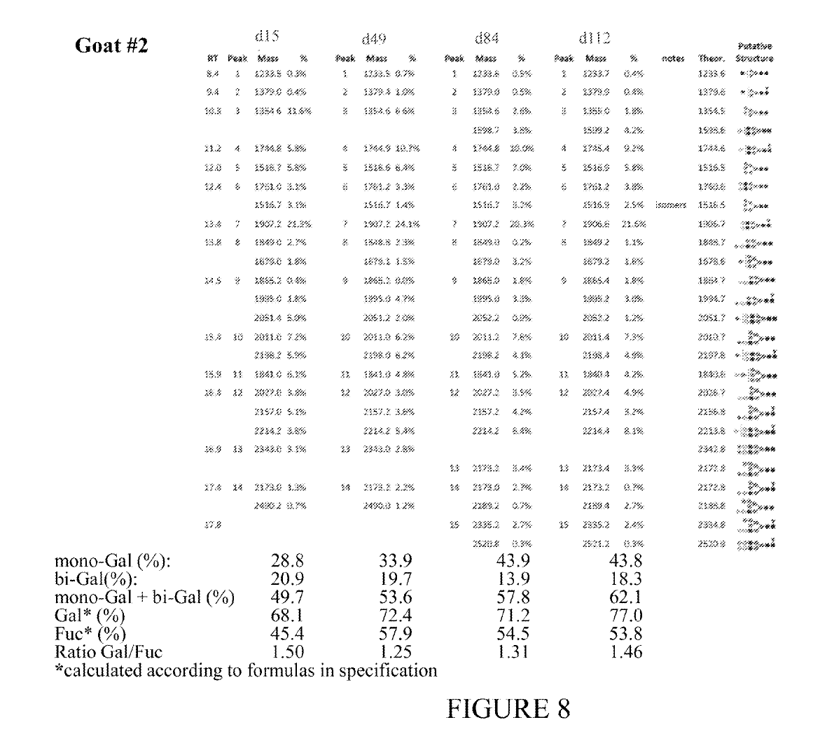

The relative percentages of all N-glycan oligosaccharides isolated from the trastuzumab antibody produced in the milk of goat #2 at day 7 of lactation are depicted in FIG. 7. FIG. 7 also tabulates the overall percentage of mono-galactosylation, percentage of bi-galactosylation, percentage of total galactosylation (mono-galactosylation+bi-galactosylation), percentage of galactosylation as calculated according to the formula provided above, percentage of fucosylation as calculated according to the formula provided above, and the ratio of galactosylation to fucosylation of trastuzumab antibodies produced in goat #2 at day 7 of lactation. FIG. 8 presents relative percentages of different N-glycan oligosaccharides isolated from the trastuzumab antibody produced in the milk of goat #2 at days 15, 49, 84, and 112 of lactation. The results are also summarized in Table 2 below:

TABLE 2
N-glycan oligosaccharides isolated from
trastuzumab antibodies from goat #2

| | day 7 | day 15 | day 49 | day 84 | day 112 |
|---|---|---|---|---|---|
| mono-Gal (%): | 23.7 | 28.8 | 33.9 | 43.9 | 43.8 |
| bi-Gal (%): | 29.1 | 20.9 | 19.7 | 13.9 | 18.3 |
| mono-Gal + bi-Gal (%) | 52.8 | 49.7 | 53.6 | 57.8 | 62.1 |
| Gal* (%) | 50.4 | 68.1 | 72.4 | 71.2 | 77.0 |
| Fuc* (%) | 47.8 | 45.4 | 57.9 | 54.5 | 53.8 |
| Ratio Gal/Fuc | 1.05 | 1.50 | 1.25 | 1.31 | 1.46 |

*calculated according to formulas in specification

### Example 2

### Glycosylation Analysis of Transgenically Produced Trastuzumab in Additional Animals

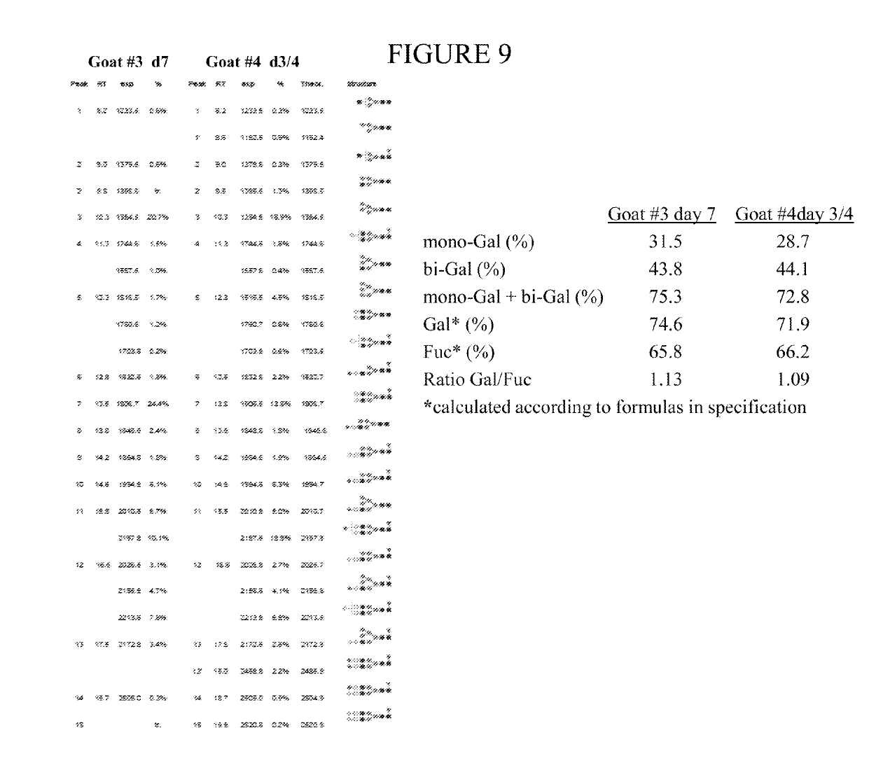

The relative percentages of different N-glycan oligosaccharides present in transgenically produced trastuzumab antibody from the milk of goat #3 on day 7 of lactation and goat #4 on day 3/4 of lactation are depicted in FIG. 9 and are also summarized in Table 3 below:

TABLE 3
Summary of data on production of trastuzumab in goats #3 and #4

| | Goat #3 day 7 | Goat #4 day 3/4 |
|---|---|---|
| mono-Gal (%) | 31.5 | 28.7 |
| bi-Gal (%) | 43.8 | 44.1 |
| mono-Gal + bi-Gal (%) | 75.3 | 72.8 |
| Gal* (%) | 74.6 | 71.9 |
| Fuc* (%) | 65.8 | 66.2 |
| Ratio Gal/Fuc | 1.13 | 1.09 |

*calculated according to formulas in specification

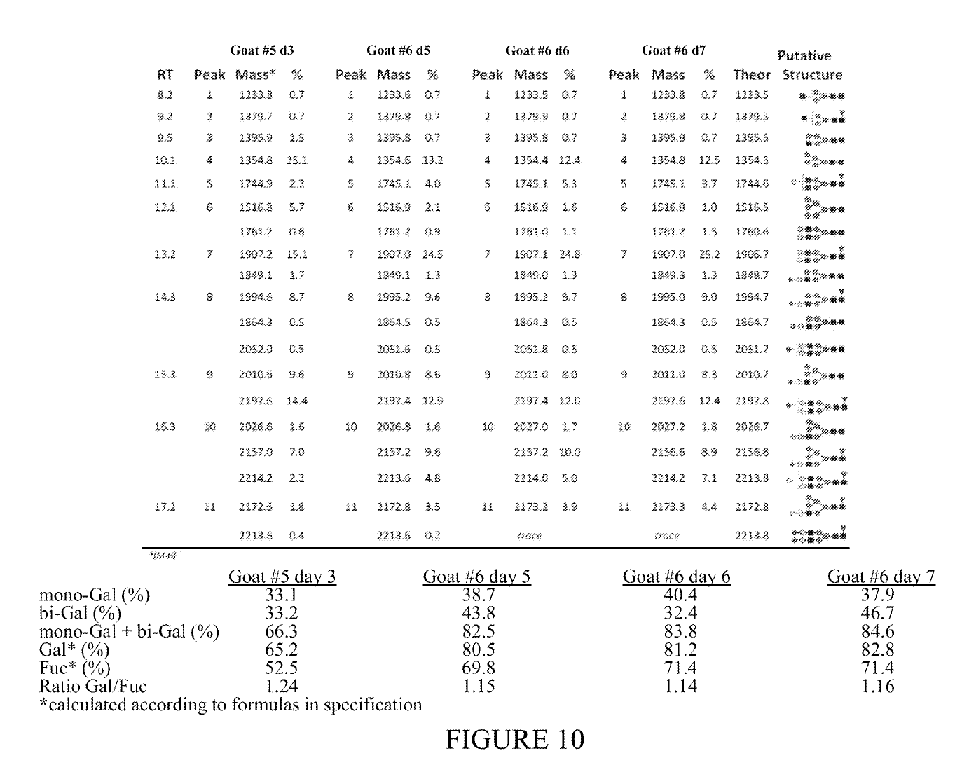

The relative percentages of different N-glycan oligosaccharides present in transgenically produced trastuzumab antibody from the milk of goat #5 on day 3 of lactation and goat #6 on days 5, 6, and 7 of lactation are depicted in FIG. 10 and are also summarized in Table 4 below:

TABLE 4
Summary of data on production of trastuzumab in goats #5 and #6

| | Goat #5 | | Goat #6 | Goat #6 |
|---|---|---|---|---|
| | day 3 | Goat #6 day 5 | day 6 | day 7 |
| mono-Gal (%) | 33.1 | 38.7 | 40.4 | 37.9 |
| bi-Gal (%) | 33.2 | 43.8 | 32.4 | 46.7 |
| mono-Gal + bi-Gal (%) | 66.3 | 82.5 | 83.8 | 84.6 |
| Gal* (%) | 65.2 | 80.5 | 81.2 | 82.8 |
| Fuc* (%) | 52.5 | 69.8 | 71.4 | 71.4 |
| Ratio Gal/Fuc | 1.24 | 1.15 | 1.14 | 1.16 |

*calculated according to formulas in specification

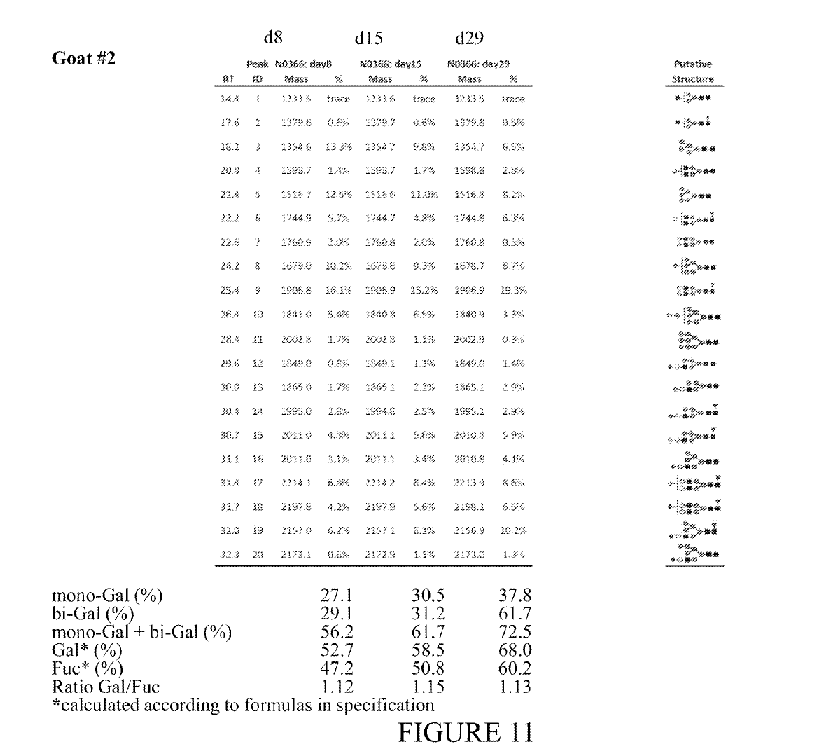

The relative percentages of different N-glycan oligosaccharides present in transgenically produced trastuzumab antibody from the milk of goat #2 on days 8, 15, and 29 of the second lactation are depicted in FIG. 11 and are also summarized in Table 5 below:

TABLE 5
Summary of data on production of trastuzumab
in goat #2 in the second lactation

| | day 8 | day 15 | day 29 |
|---|---|---|---|
| mono-Gal (%) | 27.1 | 30.5 | 37.8 |
| bi-Gal (%) | 29.1 | 31.2 | 61.7 |
| mono-Gal + bi-Gal (%) | 56.2 | 61.7 | 72.5 |
| Gal* (%) | 52.7 | 58.5 | 68.0 |
| Fuc* (%) | 47.2 | 50.8 | 60.2 |
| Ratio Gal/Fuc | 1.12 | 1.15 | 1.13 |

*calculated according to formulas in specification

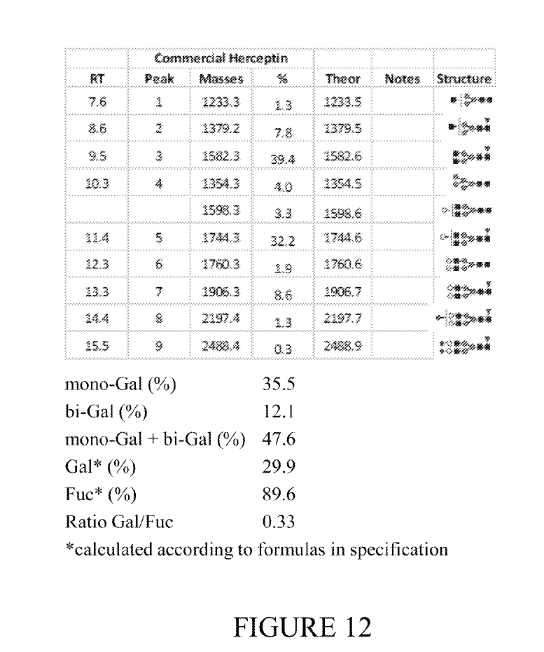

The relative percentages of different N-glycan oligosaccharides present in commercial Herceptin®/trastuzumab are depicted in FIG. 12 and are also summarized in Table 6 below:

TABLE 6
Summary of data on production of commercial Herceptin ®/trastuzumab

| | |
|---|---|
| mono-Gal (%) | 35.5 |
| bi-Gal (%) | 12.1 |
| mono-Gal + bi-Gal (%) | 47.6 |
| Gal* (%) | 29.9 |
| Fuc* (%) | 89.6 |
| Ratio Gal/Fuc | 0.33 |

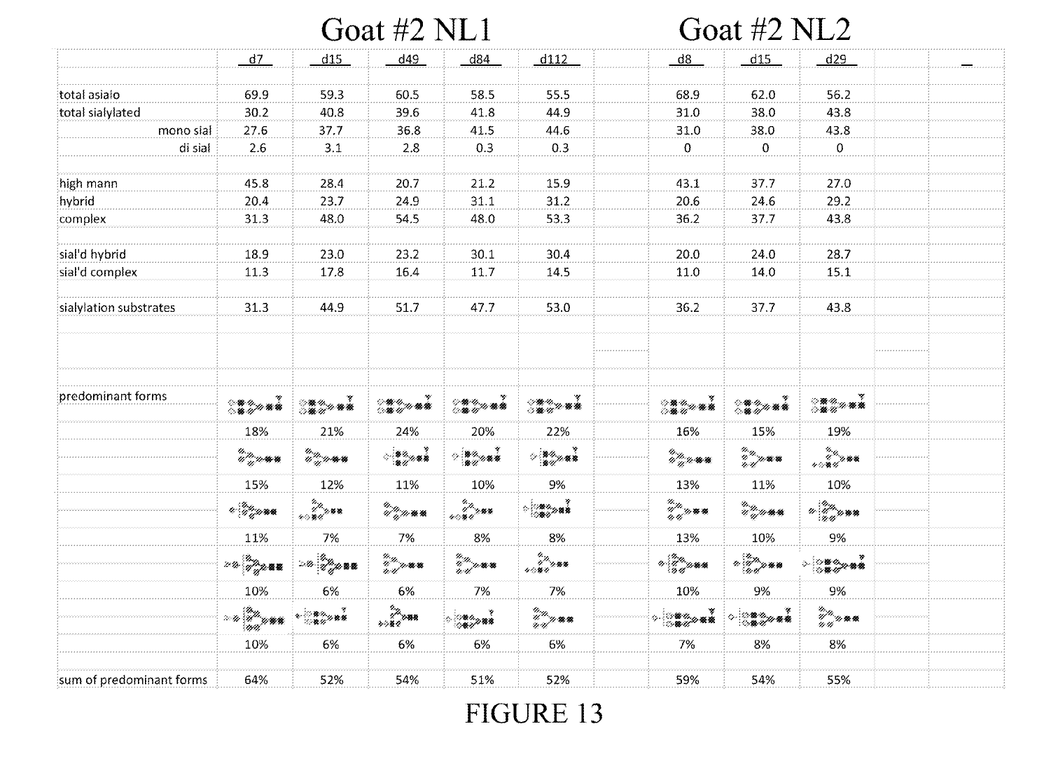

FIG. 13 shows a summary comparing the sialic acid and mannose modifications and predominant forms of trastuzumab produced by goat #2 at various days of the first lactation (NL1) and second lactation (NL2).

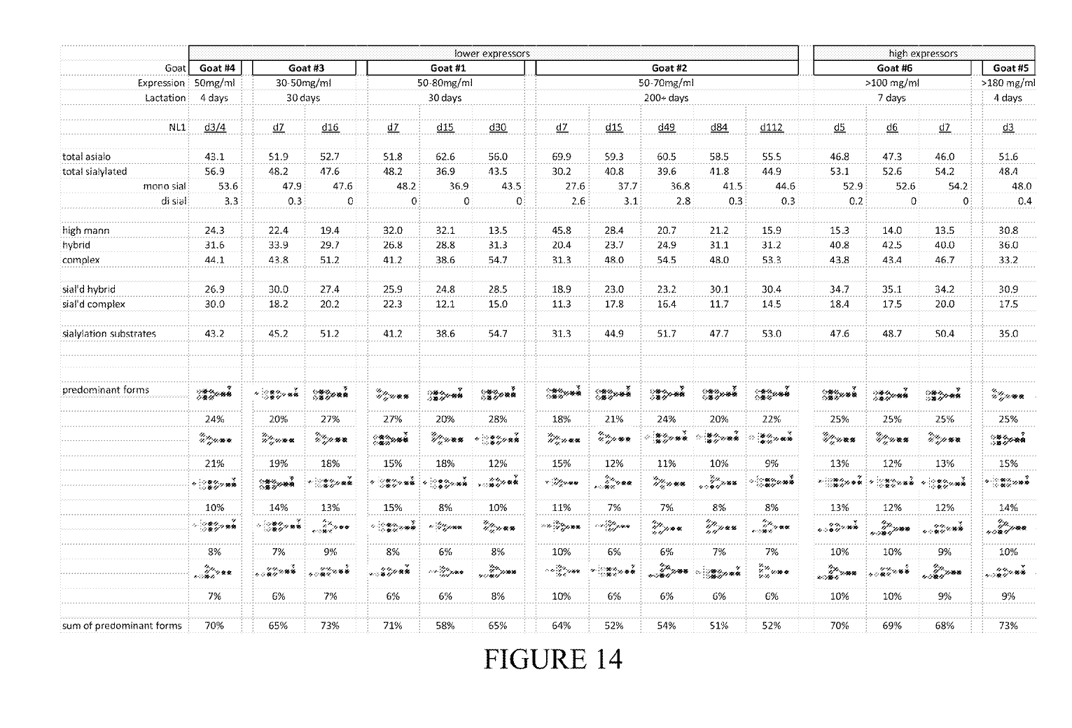

FIG. 14 shows a summary of the sialic acid and mannose modifications and predominant forms of trastuzumab produced in goats #1-6.

### Example 3

### Characterization of Transgenically Produced Trastuzumab

Functional characteristics of transgenically produced trastuzumab produced in goat milk were compared to commercial Herceptin®/Trastuzumab. Binding affinity for HER2-expressing cell lines, CD16 on NK cells and C1q were quantified. Furthermore, these antibodies were evaluated for their ability to induce lysis of HER2-expressing cell lines by Antibody-dependent Cell-Mediated Cytotoxicity (ADCC) and Complement Dependent Cytotoxicity (CDC), and for their ability to inhibit cellular proliferation.

Antigen recognition on the HER2-expressing SK-BR-3 cell line was of the same order (arbitrary dissociation constant, Kd, 2-6 μg/ml) for transgenically-produced trastuzumab Batch A, transgenically-produced trastuzumab Batch B and commercial Herceptin®/trastuzumab (Roche). Transgenically-produced trastuzumab antibodies bound to

CD16 receptor expressed by NK cells with an IC₅₀ value of 30 μg/ml for Batch A and 25 μg/ml for Batch B. In comparison, the IC₅₀ was 37 μg/ml for Herceptin®/trastuzumab indicating that binding of the transgenically-produced trastuzumab are within the range of IC₅₀ of commercial Herceptin®/trastuzumab. Similarly, the abilities of each of the tested antibodies to induce lysis of the SK-BR-3 cells by Antibody-Dependent Cell-mediated Cytotoxicity (ADCC) were comparable.

The abilities of each of the tested antibodies to mediate Complement-Dependent Cytotoxicity (CDC) activity on SK-BR-3 cells were comparable. Commercial Herceptin®/trastuzumab, transgenically-produced trastuzumab Batch A and Batch B induced 55, 55 and 50% of growth inhibition on BT-474 cells, respectively.

### Materials and Methods

Binding assay to HER2-expressing SKBR-3 cell line

### Reagents

anti-HER2 antibodies:
Herceptin® (Trastuzumab) (Roche)
Transgenically produced trastuzumab Batch A
Transgenically produced trastuzumab Batch B

Target Cells
SK-BR-3 cells

### Methods

2×10⁵ cells were incubated with 100 μl of anti-HER2 antibodies (10 μg/ml) at 4° C. for 30 minutes. After washing, humanized HER2 mAb antibodies were detected with goat anti-human (H+L) coupled with phycoerythrine (100 μl of a dilution of 1:100) and incubated at 4° C. for 30 minutes. After washing, cells were analyzed by flow cytometry (FC500, Beckman Coulter).

### Determination of Relative Dissociation Constant (Kd)—Antibody Binding to Cell Surface Antigen

### Reagents

anti-HER2 antibodies:
Herceptin® (Trastuzumab) (Roche)
Transgenically produced trastuzumab Batch A
Transgenically produced trastuzumab Batch B

### Methods

2×10⁵ cells were incubated with 100 μl of anti-HER2 antibodies coupled to Alexa 488 at different concentrations (0 to 50 μg/ml, final concentration) at 4° C. for 30 minutes. After washing, cells were analyzed by flow cytometry (FC500, Beckman Coulter).

Maximum binding (Bmax) and arbitrary dissociation constant (Kd) values were calculated using PRISM software. Arbitrary Kd expressed in μg/ml does not represent the real affinity value commonly expressed in nM, but gives an order of magnitude between the studied antibodies.

Assay to Quantify Interaction with CD16 Expressed by NK Cells

### Reagents

Antibodies:
   Herceptin® (Trastuzumab)(Roche)
   Transgenically produced trastuzumab Batch A
   Transgenically produced trastuzumab Batch B
NK effector cells from a healthy donor extracted from peripheral blood were purified by negative depletion (Miltenyl Biotec)
Target Cells
   SK-BR-3 cells

### Methods

mAb binding to CD16 expressed by NK cells was measured using a competitive assay with the anti-CD16 antibody (3G8 clone).

NK cells purified by negative depletion (Miltenyl) from the peripheral blood of healthy donors were incubated with varying concentrations (0 to 83 μg/ml) of the anti-HER2 (Herceptin® or transgenically produced trastuzumab),® with the anti-CD16 antibody 3G8 conjugated to PE (3G8-PE) at a fixed concentration. After washing, 3G8-PE bound to the CD16 receptor on the NK cells was evaluated by flow cytometry. The mean fluorescence values (MFI) observed are expressed as the percent binding, where 100% was the value observed without addition of a tested antibody that thus corresponds to maximum 3G8 binding, and 0% corresponds to the MFI in the absence of the 3G8-PE antibody. The IC₅₀, the antibody concentration required to induce 50% inhibition of 3G8 binding, was calculated for each tested antibody using PRISM software.

### Antibody-Dependent Cellular Cytotoxicity (ADCC) Assay Reagents

Antibodies:
Herceptin® (Trastuzumab)(Roche)
Transgenically produced trastuzumab Batch A
Transgenically produced trastuzumab Batch B
Anti-idiotypic FVIII (a chimeric anti-Factor VIII antibody produced by rat hybridoma YB2/0), also referred to as “Anti-id FVIII (antibody)”.

Effector cells:
NK effector cells from a healthy donor extracted from peripheral blood were purified by negative depletion (Miltenyl Biotec).

Target Cells:
SK-BR-3 cells

### Methods

SK-BR-3 target cells were plated in 96 well plate with NK cells, with E/T: 10/1 and increasing concentrations of anti-HER2 antibodies.

After 16 hours of incubation, target cells lysis induced by anti-HER2 antibodies was measured chromographically by quantifying the intracellular enzyme lactate dehydrogenase (LDH) released into the supernatant from the lysed target cells (Roche Diagnostics).

### Analysis

The percent lysis was calculated according to the following formula:

% lysis=[(ER−SR)/(100−SR)]−[(NC−SR)/(100−SR)]

Where ER and SR represent experimental and spontaneous LDH release, respectively; and NC represents natural cytotoxicity.

The results (% lysis) are expressed as a function of the antibody concentration (0-5000 ng/ml). Emax, the percentage of maximum lysis, and EC₅₀, the quantity of antibody that induces 50% of maximum lysis, were calculated using PRISM software.

### Complement-Dependent Cytotoxicity (CDC) Assay

### Reagents

Antibodies:
Herceptin® (Trastuzumab)(Roche)
Transgenically produced trastuzumab Batch A
Transgenically produced trastuzumab Batch B
Anti-id FVIII antibody

Target Cells:
SK-BR-3 cells

### Method

Targets cells were incubated with increasing concentrations of anti-HER2 antibodies (0 to 25000 ng/ml) in the presence of baby rabbit serum as a source of complement (dilution to 1/10). After 1 hour of incubation at 37° C., the quantity of LDH released into the supernatant by lysed target cells was measured by fluorimetry (Roche Applied Sciences) and used to calculate the percentage of CDC activity mediated by the tested antibodies.

### Analysis

The percent lysis was calculated according to the following formula:=

% lysis=ER−SA

ER: experimental response

SA: spontaneous activity obtained when target cell is incubated in presence of complement, without antibody.

Results are expressed as the percent of lysis as a function of the antibody concentration.

### C1q Binding Assay

### Reagents

Antibodies:
Herceptin® (Trastuzumab):
Transgenically produced trastuzumab Batch A
Transgenically produced trastuzumab Batch B
Anti-id FVIII antibody
C1q (Sigma)

Polyclonal rabbit anti-human C1q (DAKO)

Polyclonal Swine anti-rabbit IgG HRP (DAKO)

2,2′-Azino-bis(3-ethylbenzothiazoline-6-sulfonic acid) (ABTS) (Sigma)

1% SDS in PBS (Fluka)

### Method

Briefly, increasing concentrations (0 to 10 μg/ml) of antibodies anti HER2 were coated in 96-well plates overnight at 4° C. After 1 hour of saturation with PBT (PBS, BSA 1%, Tween-20 0.05%), human C1q was diluted at 2 μg/ml added and incubated for 1 h at room temperature. Then, bound C1q was recognized by applying polyclonal rabbit anti-human C1q antibody followed by polyclonal Swine anti-rabbit IgG conjugated to horse radish peroxidase (HRP). The substrate ABTS was added, and following reaction with peroxidase, a blue-green reaction product was formed that was read at wavelength 405 nm. The reaction was stopped by adding a volume of 1% SDS equal to that of the substrate in the well.

### Cell Proliferation Assay

### Reagents

Antibodies:
Herceptin® (Trastuzumab)(Roche)
Transgenically produced trastuzumab Batch A
Transgenically produced trastuzumab Batch B
Anti-id FVIII antibody

Target Cells:
BT-474 cells

### Method

Target cells were plated in 96-well plates at 1×10⁴ cells/well and cultured for 72 h at 37° C. with increasing concentrations of anti-HER2 antibodies (0 up 100 μg/ml). All dilutions were performed in the culture medium (final volume 100 μL/well).

Camptothecin (1 μg/ml), a cytotoxic quinoline alkaloid that inhibits the DNA enzyme topoisomerase I (topo I), was used as a positive control of inhibition of cellular proliferation in the same condition.

### Analysis

Cell proliferation was measured on day 3 with a colorimetric method “Cell Titer 96 Aqueous One Solution Cell Proliferation Assay” (PROMEGA) which allows determination of the number of viable cells. Briefly, the MTS substrate is bioreduced by cells into a colored formazan. The quantity of formazan product, as measured by absorbance at wavelength 490 nm, is directly proportional to the number of living cells in culture.

20 μl of MTS was added to each well. After 2 hours of incubating according to the specific cell line metabolism, the absorbance was recorded at wavelength 490 nm in a 96-well plate reader. Results are expressed as a percentage of cell growth or percentage of inhibition of proliferation, with 100% corresponding to the target cell proliferation without antibody.

### Results

### Binding to HER2-Expressing Cell Line

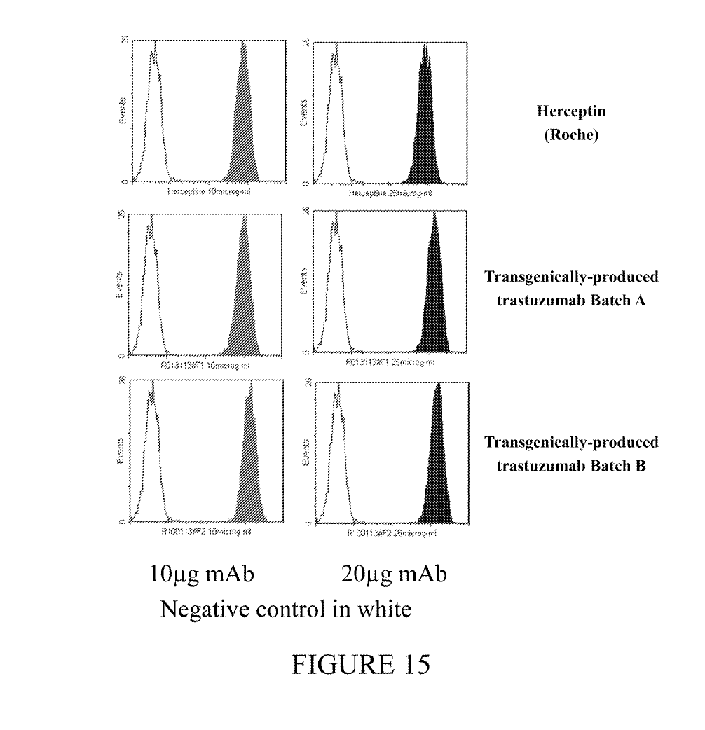

As presented in FIG. 15, both commercial Herceptin®/trastuzumab and two batches of transgenically-produced trastuzumab, A and B, recognized and bound to the SK-BR-3 cell line that expresses HER2 as compared to the negative control, shown in white.

### Determination of Relative Kd

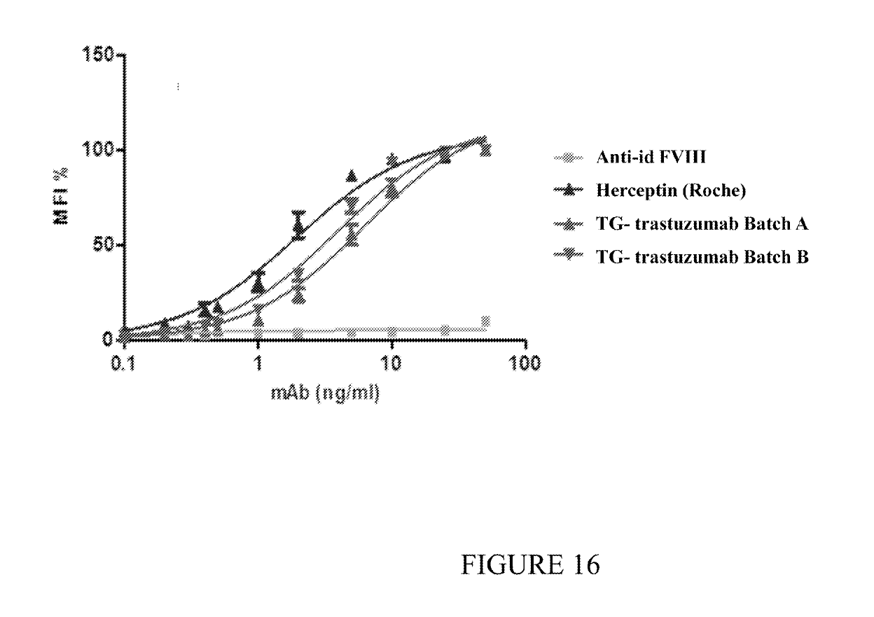

Four independent binding assays were performed and the mean of the assays are presented in FIG. 16 and the corresponding data are presented in Tables 7 and 8. Binding of anti-HER2 antibodies to membrane HER2 expressed SK-BR-3 cells is expressed as the mean of fluorescence intensity (MFI) for each antibody concentration tested (0-50 μg/ml). The arbitrary Kd does not represent the real affinity value (nM), but gives a comparable order of magnitude between the studied antibodies. As presented in Table 7, the mean arbitrary Kd (concentration giving 50% of the plateau value) are 6.16 μg/ml, 3.99 μg/ml and 1.95 μg/ml for transgenically-produced trastuzumab Batch A, transgenically-produced trastuzumab Batch B and commercial Herceptin®/trastuzumab, respectively. As the IgG content of Herceptin was not determined and may affect Kd determination, these experiments show that commercial Herceptin®/trastuzumab and transgenically-produced trastuzumab bind similarly to HER2 expressing cells.

TABLE 7
Summary of mean Bmax and Kd values corresponding to FIG. 16.

| | | | Transgenic | Transgenic |
|---|---|---|---|---|
| | Irrelevant | Herceptin | trastuzumab | trastuzumab |
| | Ab | (Roche) | Batch A | Batch B |
| Bmax | NA | 109 | 119 | 116 |
| Kd | NA | 1.95 | 6.16 | 3.99 |

Results derived from an average of 4 experiments are expressed as MFI and μg/ml respectively.

TABLE 8
Data from three experiments evaluating Bmax and Kd values

| | | | | | | | | Transgenic | | | | Transgenic | | | |
|---|---|---|---|---|---|---|---|---|---|---|---|---|---|---|---|
| | | | | | | | | trastuzumab | | | | Trastuzumab | | | |
| mAB ng/ml | Anti id FvIII | | | | Herceptin (Roche) | | | Batch A | | | | Batch B | | | |
| 0 | 5 | 9 | 7 | 3 | 4 | 9 | 2 | 2 | 4 | 3 | 1 | 2 | 6 | 3 | 1 |
| 0.1 | 4 | 6 | 7 | 2 | 6 | 7 | 4 | 3 | 3 | 2 | 1 | 3 | 3 | 2 | 2 |
| 0.2 | 4 | 0 | 4 | 2 | 8 | 12 | 7 | 3 | 4 | 3 | 2 | 4 | 4 | 4 | 3 |
| 0.3 | 4 | 6 | 5 | 3 | 7 | 9 | 6 | 3 | 4 | 3 | 2 | 3 | 4 | 3 | 3 |
| 0.4 | 4 | 0 | 4 | 3 | 14 | 23 | 12 | 5 | 7 | 5 | 4 | 7 | 9 | 6 | 7 |
| 0.5 | 4 | 4 | 7 | 2 | 18 | 20 | 15 | 6 | 8 | 5 | 5 | 8 | 10 | 8 | 8 |
| 1 | 4 | 4 | 5 | 2 | 24 | 40 | 29 | 10 | 15 | 10 | 10 | 13 | 21 | 14 | 16 |
| 2 | 4 | 4 | 5 | 3 | 56 | 74 | 52 | 23 | 34 | 20 | 19 | 31 | 44 | 32 | 32 |
| 5 | 4 | 5 | 5 | 2 | 84 | 89 | 87 | 53 | 71 | 53 | 46 | 68 | 81 | 69 | 65 |
| 10 | 4 | 5 | 5 | 3 | 95 | 93 | 97 | 80 | 91 | 81 | 69 | 94 | 95 | 94 | 91 |
| 25 | 5 | 6 | 6 | 5 | 94 | 96 | 100 | 99 | 101 | 99 | 97 | 98 | 98 | 98 | 102 |
| 50 | 7 | 7 | 16 | 10 | 100 | 100 | 100 | 100 | 100 | 100 | 100 | 100 | 100 | 100 | 100 |

Interaction with CD16 Expressed by NK Cells

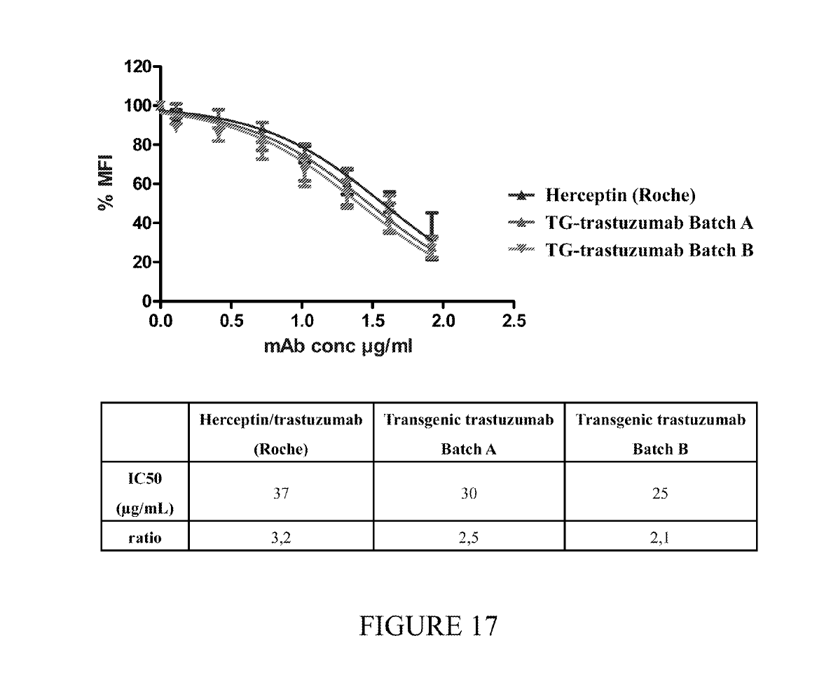

The ability of each anti-HER2 antibody to bind CD16 expressed by NK cells was studied in a competitive assay using 3G8-PE mAb. FIG. 17 presents the competitive CD16 binding data for commercial Herceptin®, transgenically-produced trastuzumab Batch A and transgenically-produced trastuzumab Batch B wherein CD16+ NK cells were incubated with increasing amount of anti-HER2 antibody together with PE-conjugated anti-CD16+ 3G8 mAb. The percentages of 3G8 binding were calculated as described in the Materials and Methods section. The amount of mAb required to reduce the binding of 3G8-PE mAb by 50% were 37, 30 and 25 μg/ml for commercial Herceptin®, transgenically-produced trastuzumab Batch A and transgenically-produced trastuzumab Batch B, respectively (FIG. 17, Table 9). Binding of transgenically-produced trastuzumab to CD16 is similar to that of commercial Herceptin®

TABLE 9
Summary of IC50 (antibody concentration required to induce
50% of inhibition of 3G8 binding), corresponding to FIG. 17,
after modeling of the curve by the PRISM software.

| | | | Transgenic |
|---|---|---|---|
| | Herceptin/trastuzumab | Transgenic trastuzumab | trastuzumab |
| | (Roche) | Batch A | Batch B |
| IC50 | 37 | 30 | 25 |
| (μg/mL) | | | |
| ratio | 3.2 | 2.5 | 2.1 |

TABLE 10
Data from three experiments evaluating anti-HER2
antibody binding to CD16

| log[Ab] | | | | |
|---|---|---|---|---|
| μg/ml | Herceptin (Roche) | | | |
| 0 | 100 | 100 | 100 | 100 |
| 0.11 | 87 | 91 | 104 | 95 |
| 0.41 | 89 | 91 | 100 | 95 |
| 0.72 | 88 | 86 | 90 | 89 |
| 1.02 | 64 | 83 | 72 | 80 |
| 1.32 | 46 | 74 | 55 | 69 |
| 1.62 | 40 | 62 | 44 | 57 |
| 1.92 | 10 | 48 | ND | 42 |

| | Transgenic | | | | Transgenic | | | |
|---|---|---|---|---|---|---|---|---|
| log[Ab] | trastuzumab | | | | trastuzumab | | | |
| μg/ml | Batch A | | | | Batch B | | | |
| 0 | 100 | 100 | 100 | 100 | 100 | 100 | 100 | 100 |
| 0.11 | 98 | 107 | 88 | 96 | 84 | 92 | 85 | 92 |
| 0.41 | 98 | 102 | 80 | 93 | 80 | 93 | 79 | 89 |
| 0.72 | 87 | 98 | 64 | 88 | 74 | 86 | 66 | 82 |
| 1.02 | 75 | 89 | 44 | 75 | 58 | 84 | 50 | 74 |
| 1.32 | 62 | 76 | 32 | 63 | 47 | 71 | 40 | 61 |
| 1.62 | 45 | 57 | 21 | 47 | 37 | 59 | 23 | 61 |
| 1.92 | 32 | 37 | 13 | 30 | 25 | 37 | 14 | 32 |

### ADCC Evaluation

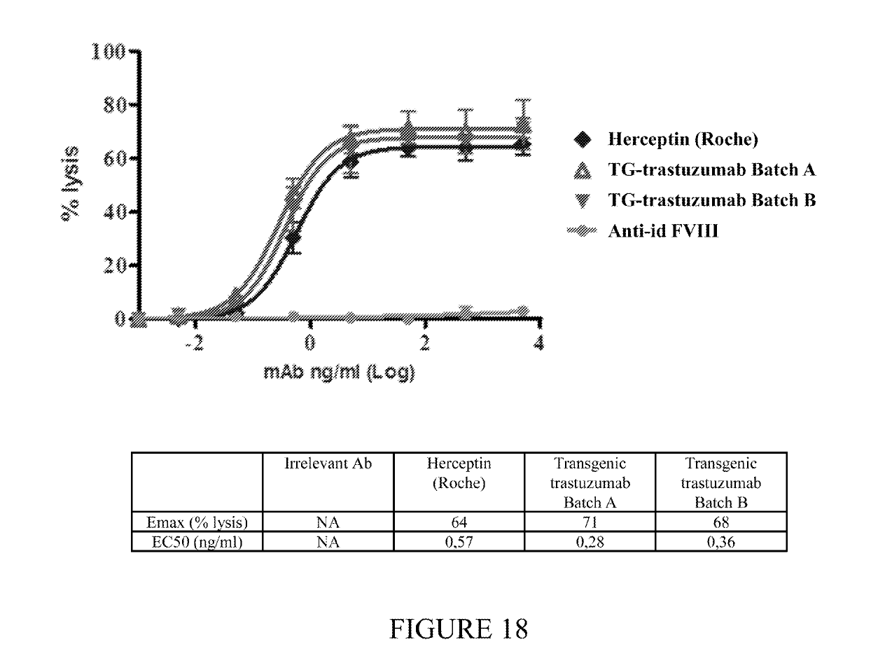

As presented in FIG. 18, the maximal lysis of SK-BR-3 cells induced by commercial Herceptin®, transgenically produced trastuzumab Batch A and transgenically produced trastuzumab Batch B was 64, 71, and 68%, respectively. The data are presented as the mean of three independent experiments, and percentages of cell lysis are expressed as a function of the antibody concentration (0-5000 ng/ml). EC₅₀ values, the half maximal effective concentration, for commercial Herceptin®, transgenically produced trastuzumab Batch A and transgenically produced trastuzumab Batch B antibodies were 0.57, 0.28, and 0.36 ng/ml, respectively (Table 11). These data indicated that the ADCC activities for the three tested anti-HER2 antibodies were very similar to the binding of CD16 expressed by NK cells. Anti-Id FVIII antibody was used as a negative control and does not mediate significant cell lysis.

TABLE 11
Summary of calculated of Emax (maximum lysis) and EC₅₀
(antibody concentration required to obtain 50% of Emax)
after modeling (sigmoid) the curve with PRISM software

| | | | Transgenic | Transgenic |
|---|---|---|---|---|
| | | Herceptin | trastuzumab | trastuzumab |
| | Irrelevant Ab | (Roche) | Batch A | Batch B |
| Emax (% lysis) | NA | 64 | 71 | 68 |
| EC50 (ng/ml) | NA | 0.57 | 0.28 | 0.36 |

TABLE 12
Data from three experiments evaluating ADCC activity of anti-HER2 antibodies

| | | | | Transgenic | | | Transgenic | | | | | |
|---|---|---|---|---|---|---|---|---|---|---|---|---|
| mAb ng/ml | Herceptin | | | trastuzumab | | | trastuzumab | | | | | |
| (Log) | Roche | | | Batch A | | | Batch B | | | Anti id-FVIII | | |
| −3 | 0 | 0 | 0 | 0 | 0 | 0 | 0 | 0 | 0 | 0 | 0 | 0 |
| −2.301 | 0 | 0 | 1 | 0 | 3 | 1 | 0 | 4 | 0 | 0 | 2 | 0 |
| −1.301 | 0 | 5 | 2 | 10 | 12 | 4 | 0 | 12 | 2 | 0 | 2 | 0 |
| −0.301 | 25 | 42 | 24 | 52 | 53 | 36 | 27 | 55 | 41 | 0 | 3 | 0 |
| 0.699 | 53 | 70 | 53 | 70 | 74 | 57 | 47 | 77 | 65 | 0 | 1 | 0 |
| 1.699 | 63 | 70 | 59 | 82 | 71 | 61 | 61 | 74 | 68 | 0 | 0 | 0 |
| 2.699 | 66 | 71 | 55 | 84 | 70 | 56 | 64 | 69 | 65 | 0 | 9 | 0 |
| 3.699 | 73 | 64 | 59 | 88 | 74 | 56 | 73 | 77 | 64 | 0 | 8 | 4 |

### CDC Evaluation

The CDC activity on SK-BR-3 cells mediated by the anti-HER-2 antibodies was evaluated. The results indicate that CDC activity induced by the commercial Herceptin® or transgenically-produced trastuzumab on SK-BR-3 cells are similar

### C1q Binding

The ability of transgenically-produced trastuzumab and commercial Herceptin®/trastuzumab to bind to C1q was evaluated by ELISA. Results indicate that commercial Herceptin®/trastuzumab and transgenically-produced trastuzumab bind to human C1q, as presented in Table 13.

TABLE 13
Data from one representative C1q binding experiment

| | | OD. 405 nm | | | |
|---|---|---|---|---|---|
| | mAb conc μg/ml | Well 1 | Well2 | Mean | SD |
| Herceptin | 10 | 0.836 | 0.846 | 0.841 | 0.007 |
| (Roche) | 5 | 0.795 | 0.815 | 0.805 | 0.014 |
| | 2.5 | 0.783 | 0.801 | 0.792 | 0.013 |
| | 1.25 | 0.816 | 0.714 | 0.765 | 0.072 |
| | 0.625 | 0.433 | 0.357 | 0.395 | 0.054 |
| | 0.3125 | 0.218 | 0.223 | 0.221 | 0.004 |
| | 0.15625 | 0.165 | 0.153 | 0.159 | 0.008 |
| | 0 | 0.077 | 0.062 | 0.070 | 0.011 |
| Transgenic | 10 | 0.536 | 0.545 | 0.541 | 0.006 |
| trastuzumab | 5 | 0.498 | 0.478 | 0.488 | 0.014 |
| Batch A | 2.5 | 0.589 | 0.547 | 0.568 | 0.030 |
| | 1.25 | 0.534 | 0.559 | 0.547 | 0.018 |
| | 0.625 | 0.255 | 0.29 | 0.273 | 0.025 |
| | 0.3125 | 0.19 | 0.189 | 0.190 | 0.001 |
| | 0.15625 | 0.123 | 0.143 | 0.133 | 0.014 |
| | 0 | 0.056 | 0.058 | 0.057 | 0.001 |
| Transgenic | 10 | 0.708 | 0.669 | 0.689 | 0.028 |
| trastuzumab | 5 | 0.668 | 0.648 | 0.658 | 0.014 |
| Batch B | 2.5 | 0.599 | 0.575 | 0.587 | 0.017 |
| | 1.25 | 0.381 | 0.299 | 0.340 | 0.058 |
| | 0.625 | 0.186 | 0.163 | 0.175 | 0.016 |
| | 0.3125 | 0.15 | 0.144 | 0.147 | 0.004 |
| | 0.15625 | 0.099 | 0.107 | 0.103 | 0.006 |
| | 0 | 0.051 | 0.058 | 0.055 | 0.005 |

### Cell Proliferation Assay

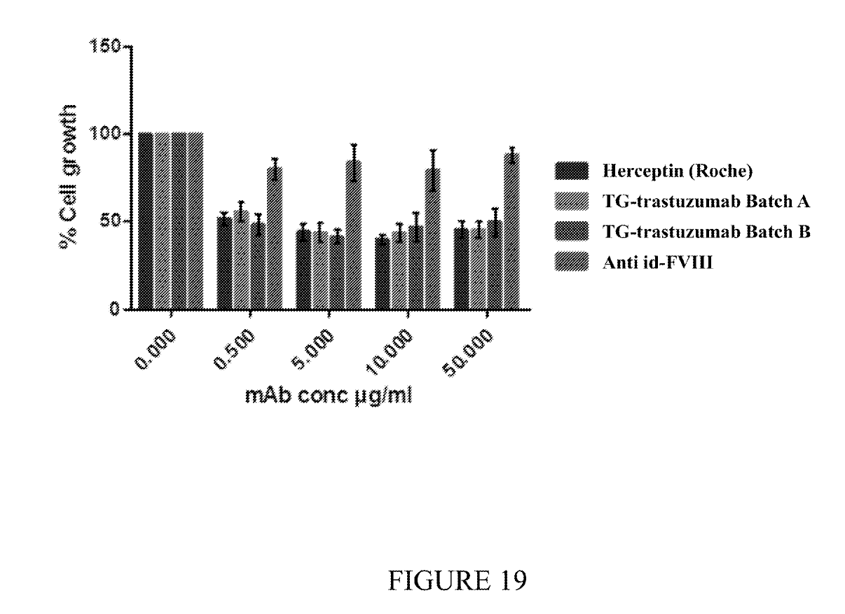

The anti-HER2 antibodies were evaluated for their ability to inhibit proliferation of BT-474 cells. Percent of target cell proliferation was measured in the presence of humanized anti-HER2 antibodies or a negative control antibody (anti-id FVIII) following 72 h and presented as the mean of three assays. A value of 100% corresponds to the amount of cell proliferation obtained without an antibody. Data are presented in FIG. 19 and Table 14.

The negative control antibody (anti-id FVIII) induced less than 10% of inhibition of BT-474 cell proliferation. In contrast, incubation of BT-474 cells with commercial Herceptin®/trastuzumab, transgenically-produced trastuzumab Batch A, and transgenically-produced trastuzumab Batch B resulted in 55, 55, and 50% of growth inhibition, respectively.

TABLE 14
Cell proliferation data corresponding to FIG. 19

| | | | | Transgenic | | | Transgenic | | | | | |
|---|---|---|---|---|---|---|---|---|---|---|---|---|
| mAb | Herceptin | | | trastuzumab | | | trastuzumab | | | | | |
| μg/ml | (Roche) | | | Batch A | | | Batch A | | | anti id FVIII | | |
| 0 | 100 | 100 | 100 | 100 | 100 | 100 | 100 | 100 | 100 | 100 | 100 | 100 |
| 0.5 | 58 | 52 | 45 | 63 | 59 | 45 | 60 | 39 | 46 | 74 | 92 | 74 |
| 5 | 50 | 48 | 34 | 53 | 44 | 34 | 49 | 39 | 36 | 94 | 94 | 63 |
| 10 | 42 | 43 | 34 | 51 | 46 | 34 | 58 | 52 | 31 | 85 | 96 | 57 |
| 50 | 52 | 48 | 36 | 51 | 49 | 36 | 62 | 52 | 35 | 95 | 89 | 80 |

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. An anti-HER2 antibody, wherein the antibody is highly galactosylated.

2. The antibody of claim 1, wherein the antibody is highly fucosylated.

3. The antibody of claim 1 or claim 2, wherein the antibody comprises mono-galactosylated N-glycans.

4. The antibody of any one of claims 1-3, wherein the antibody comprises bi-galactosylated N-glycans.

5. The antibody of any one of claims 1-4, wherein the heavy chain of the antibody comprises SEQ ID NO:1, and wherein the light chain of the antibody comprises SEQ ID NO:2.

6. The antibody of any one of claims 1-5, wherein the antibody is trastuzumab.

7. The antibody of any one of claims 1-6, wherein the antibody is produced in mammary epithelial cells of a non-human mammal.

8. The antibody of any one of claims 1-7, wherein the antibody is produced in a transgenic non-human mammal.

9. The antibody of claim 8 or claim 9, wherein the non-human mammal is a goat, sheep, bison, camel, cow, pig, rabbit, buffalo, horse, rat, mouse or llama.

10. The antibody of claim 9, wherein the non-human mammal is a goat.

11. A composition comprising the antibody of any one of claims 1-10, further comprising milk.

12. A composition comprising the antibody of any one of claims 1-11, further comprising a pharmaceutically-acceptable carrier.

13. A composition, comprising:
a population of antibodies,
wherein the antibody is an anti-HER2 antibody, and
wherein the level of galactosylation of the antibodies in the population is at least 50%.

14. The composition of claim 13, wherein the level of galactosylation of the antibodies in the population is at least 60%.

15. The composition of claim 13, wherein the level of galactosylation of the antibodies in the population is at least 70%.

16. The composition of any one of claims 13-15, wherein the level of fucosylation of the antibodies in the population is at least 50%.

17. The composition of any one of claims 13-15, wherein the level of fucosylation of the antibodies in the population is at least 60%.

18. The composition of any one of claims 13-17, wherein the population comprises antibodies that comprise mono-galactosylated N-glycans.

19. The composition of any one of claims 13-18, wherein the population comprises antibodies that comprise bi-galactosylated N-glycans.

20. The composition of any one of claims 13-19, wherein the ratio of the level of galactosylation of the antibodies in the population to the level of fucosylation of the antibodies in the population is between 1.0 and 1.4.

21. The composition of any one of claims 13-20, wherein at least 25% of the antibodies in the population comprise bi-galactosylated N-glycans and at least 25% of the antibodies in the population comprise mono-galactosylated N-glycans.

22. The composition of any one of claims 13-21, wherein the heavy chain of the antibody comprises SEQ ID NO:1, and wherein the light chain of the antibody comprises SEQ ID NO:2.

23. The composition of any one of claims 13-22, wherein the antibody is trastuzumab.

24. The composition of any one of claims 13-23, wherein the antibody is produced in mammary epithelial cells of a non-human mammal.

25. The composition of any one of claims 13-24, wherein the antibody is produced in a transgenic non-human mammal.

26. The composition of claim 24 or claim 25, wherein the non-human mammal is a goat, sheep, bison, camel, cow, pig, rabbit, buffalo, horse, rat, mouse or llama.

27. The composition of claim 26, wherein the non-human mammal is a goat.

28. The composition of any one of claims 13-27, wherein the composition further comprises milk.

29. The composition of any one of claims 13-28, wherein the composition further comprises a pharmaceutically-acceptable carrier.

30. The composition of any one of claims 24-29, wherein the population of antibodies has an increased level of complement dependent cytotoxicity (CDC) activity when compared to a population of antibodies not produced in mammary gland epithelial cells.

31. The composition of any one of claims 24-30, wherein the population of antibodies has an increased level of antibody-dependent cellular cytotoxicity (ADCC) activity when compared to a population of antibodies not produced in mammary gland epithelial cells.

32. The composition of any one of claims 30-31, wherein the population of antibodies not produced in mammary gland epithelial cells is produced in cell culture.

33. The composition of any one of claims 30-32, wherein the level of galactosylation of the antibodies not produced in mammary gland epithelial cells is 50% or lower when compared to the level of galactosylation of the antibodies produced in mammary gland epithelial cells.

34. The composition of any one of claims 30-32, wherein the level of galactosylation of the antibodies not produced in mammary gland epithelial cells is 30% or lower when compared to the level of galactosylation of the antibodies produced in mammary gland epithelial cells.

35. The composition of any one of claims 30-32, wherein the level of galactosylation of the antibodies not produced in mammary gland epithelial cells is 10% or lower when compared to the level of galactosylation of the antibodies produced in mammary gland epithelial cells.

36. A method for producing a population of antibodies, comprising:
expressing the population of antibodies in mammary gland epithelial cells of a non-human mammal such that a population of antibodies is produced,
wherein the antibody is an anti-HER2 antibody,
wherein the level of galactosylation of the antibodies in the population is at least 50%.

37. The method of claim 36, wherein the mammary gland epithelial cells are in culture and are transfected with a nucleic acid that comprises a sequence that encodes the antibody.

38. The method of claim 36, wherein the mammary gland epithelial cells are in a non-human mammal engineered to express a nucleic acid that comprises a sequence that encodes the antibody in its mammary gland.

39. The method of claim 37 or claim 38, wherein the nucleic acid comprises SEQ ID NO:3 and SEQ ID NO:4.

40. The method of any of claims 36-39, wherein the mammary gland epithelial cells are goat, sheep, bison, camel, cow, pig, rabbit, buffalo, horse, rat, mouse or llama mammary gland epithelial cells.

41. The method of claim 40, wherein the mammary gland epithelial cells are goat mammary gland epithelial cells.

42. Mammary gland epithelial cells that produce the antibody of any one of claims 1-12 or the population of antibodies of the compositions of any one of claims 13-35.

43. A transgenic non-human mammal comprising the mammary gland epithelial cells of claim 42.

44. A method comprising administering the antibody of any one of claims 1-10 or the composition of any one claims 11-35, to a subject in need thereof.

45. The method of claim 44, wherein the subject has cancer.

46. The method of claim 45, wherein the cancer is a HER2+ cancer.

47. The method of claim 46, wherein the HER2+ cancer is breast, ovarian, stomach or uterine cancer.

48. A monoclonal anti-HER2 antibody composition comprising monoclonal anti-HER2antibodies having glycan structures on the Fc glycosylation sites (Asn297, EU numbering),
wherein said glycan structures have a galactose content of more than 60%.


SEQUENCE LISTING (4 sequences)

SEQ ID NO: 1 - protein, 469 aa, artificial sequence
  synthetic polypeptide
MEFGLSWLFLVAILKGVQCEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 236 aa, artificial sequence
  synthetic polypeptide
MDMRVPAQLLGLLLLWLRGARCDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - DNA, 1413 bp, artificial sequence
  synthetic polynucleotide
atggagttcggcctgagctggctgttcctggtggccatcctgaagggcgtgcagtgcgaggtgcagctggtcgagagcggaggaggactggtccagcctggcggcagcctgagactgagctgcgccgccagcggcttcaacatcaaggacacctacatccactgggtgcgccaggctccagggaaagggctcgaatgggtggccaggatctaccccaccaacggctacaccagatacgccgacagcgtgaagggcaggttcaccatcagcgccgacaccagcaagaacaccgcctacctgcagatgaacagcctgagggccgaggacaccgccgtgtactactgcagcagatggggtggggatggcttctacgccatggactactgggggcagggcacactggtcacagtctccagcgccagcaccaagggccccagcgtgttccccctggctccttcctctaaatccacaagcggcggcaccgctgccctgggctgcctggtgaaggactacttccccgagcccgtgaccgtgtcttggaactctggcgccctgacctccggcgtgcacaccttccccgccgtgctgcagagcagcggcctgtacagcctgagcagcgtggtgaccgtgccctcttcctctctcggaacacagacctacatctgcaacgtgaaccacaagcccagcaacaccaaggtggacaagaaggtggagcccaagagctgcgacaagacccatacatgtcctccctgtcctgctcctgagctgctgggcggaccctccgtgttcctgttcccccccaagcccaaggacaccctgatgatcagcaggacccccgaggtgacctgcgtggtggtggacgtgtcccacgaggaccctgaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagcccccatcgaaaagaccatcagcaaggccaagggccagcctcgcgagccccaggtgtacaccctgcccccctcccgcgacgagctgaccaagaaccaggtgtccctgacctgtctggtgaagggcttctaccccagcgatatcgccgtggagtgggagagcaacggccagcccgagaacaactacaagaccaccccccctgtgctggacagcgacggcagcttcttcctgtacagcaagctgaccgtggacaagagcaggtggcagcagggaaatgtcttttcctgttccgtcatgcatgaagctctgcacaaccactacacccagaagtccctgagcctgagccccggcaagtgatag

SEQ ID NO: 4 - DNA, 711 bp, artificial sequence
  synthetic polynucleotide
atggacatgagagtgcctgcccagctcctgggactcctcctcctgtggctcaggggtgctcgctgcgatatccagatgactcagtctccttcttccctctccgccagcgtgggcgacagagtgaccatcacctgcagggccagccaggacgtgaacaccgccgtggcctggtatcagcagaagcccggcaaggcccccaagctgctgatctacagcgccagcttcctgtacagcggcgtgcccagcaggttcagcggcagcagaagcggcaccgacttcaccctgaccatcagcagcctgcagcccgaggacttcgccacctactactgccagcagcactacaccaccccccccaccttcggccagggcaccaaggtggagatcaagaggaccgtggccgctcccagcgtgttcatcttcccccccagcgacgagcagctgaagtccggcaccgcctccgtggtgtgcctgctgaacaacttctacccccgcgaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgcgaggtgacccaccagggcctgtccagccccgtgaccaagagcttcaacaggggcgagtgctga